(19) United States
(12) Patent Application Publication
(10) Pub. No.: US 2014/0186468 A1
(43) Pub. Date: Jul. 3, 2014

(54) DIAGNOSING SUBSETS OF TRIPLE-NEGATIVE BREAST CANCER

(71) Applicants: Susana GONZALO, St. Louis, MO (US); Adriana DUSSO, Lleida (ES); Xavier MATIAS-GUIU, Lleida (ES); Anna Novell ALVAREZ, Tarrega (ES); Ignacio GONZALEZ-SUAREZ, Corcelles (CH); David GROTSKY, St. Louis, MO (US)

(72) Inventors: Susana GONZALO, St. Louis, MO (US); Adriana DUSSO, Lleida (ES); Xavier MATIAS-GUIU, Lleida (ES); Anna Novell ALVAREZ, Tarrega (ES); Ignacio GONZALEZ-SUAREZ, Corcelles (CH); David GROTSKY, St. Louis, MO (US)

(21) Appl. No.: 14/099,266

(22) Filed: Dec. 6, 2013

Related U.S. Application Data
(60) Provisional application No. 61/734,836, filed on Dec. 7, 2012.

(51) Int. Cl. C12Q 1/68; G01N 33/574

(52) U.S. Cl. CPC C12Q 1/6886; G01N 33/57415
USPC 424/649; 435/7.92; 435/7.1; 435/6.12; 514/167; 514/46

(57) ABSTRACT

The present invention provides a unique combination of biomarkers that identify triple-negative breast cancer (TNBC) patient subpopulations. Such subpopulations will have differential responses to therapies, and thus treatments can be tailored to particular patients.

This application claims benefit of priority to U.S. Provisional Application Ser. No. 61/734,836, filed Dec. 7, 2012, the entire contents of which are hereby incorporated by reference.

Pursuant to 37 C.F.R. 1.821(c), a sequence listing is submitted herewith as an ASCII compliant text file named “USTLP0030US.txt” created on Dec. 5, 2013 and having a size of ˜1.3 kilobytes. The content of the aforementioned file is hereby incorporated by reference in its entirety.

This invention was made with government support under Grant No. BC110089 awarded by the Department of Defense and Grant No. RO1 GM094513-01 awarded by the National Institute of General Medical Sciences. The government has certain rights in the invention.

### BACKGROUND OF THE INVENTION

I. Field of the Invention

The present invention relates generally to the fields of oncology, molecular biology, and medicine. More particularly, the invention relates to use of certain biomarker proteins that are dysregulated in so-called “triple-negative” breast cancer as both diagnostic and therapeutic targets.

II. Description of Related Art

BRCA1 is a well-established tumor suppressor, and women carrying germline mutations in BRCA1 have a high risk of developing breast and ovarian cancer (Neuhausen and Marshall, 1994; Wooster and Weber, 2003). Tumors that arise often lack expression of estrogen and progesterone receptors and Her2, being classified as “Triple-Negative” breast cancers (TNBC) (Turner and Reis-Filho, 2006). BRCA1 participates in DNA double-strand breaks (DSBs) repair, S- and G2/M-phase cell-cycle checkpoints after damage, control of centrosome numbers, maintenance of heterochromatin, and transcriptional regulation of several genes (Mullan et al., 2006; Scully and Livingston, 2000; Zhu et al., 2011). In addition, BRCA1 function is linked to epigenetic mechanisms such as DNA methylation and miRNA biogenesis (Kawai and Amano, 2012; Shukla et al., 2010; Tanic et al., 2011).

Recruitment of BRCA1 to DNA DSBs facilitates repair by homologous recombination (HR), and loss of BRCA1 results in genomic instability characterized by unrepaired DNA breaks and complex chromosomal rearrangements that compromise cell viability (Moynahan et al., 1999; Scully et al., 1997a; Snouwaert et al., 1999). As such, BRCA1 knock-out mice and mice carrying a BRCA1 deletion mutant (BRCA1Δ11/Δ11) are embryonic lethal (Evers and Jonkers, 2006; Xu et al., 2001). While lethality in BRCA1Δ11/Δ11 mice can be rescued by abrogation of ATM, Chk2 or p53, these mice ultimately develop tumors and premature aging (Cao et al., 2006). Recently, loss of the DNA repair factor 53BP1 was shown to rescue embryonic lethality in BRCA1-deficient mice while maintaining a low incidence of tumorigenesis and normal aging (Cao et al., 2009). This is in contrast to 53BP1 knockout mice which are cancer prone (Ward et al., 2003), suggesting that 53BP1 contributes to the developmental defects of BRCA1-deficient mice and that 53BP1 loss has different consequences for cancer and aging in the context of BRCA1 proficiency or deficiency.

Loss of 53BP1 promotes viability of BRCA1-deficient cells by rescuing the loss of BRCA1 function in HR (Bouwman et al., 2010; Bunting et al., 2010; Cao et al., 2009). Importantly, downregulation of 53BP1 was observed in human BRCA1-related and TNBC, and was suggested to allow these tumors to overcome the genomic instability caused by HR defects (Bouwman et al., 2010). 53BP1 facilitates DNA DSBs repair by non-homologous end joining (NHEJ) (Fernandez-Capetillo et al., 2002; Schultz et al., 2000; Wang et al., 2002; Xie et al., 2007), and also affects HR via inhibition of BRCA1-mediated DSB end-resection (Bunting et al., 2010). The current model is that BRCA1 deficiency hinders end resection of DSBs by CtIP and the MRN (Mrel1/Rad50/Nbs1) complex, an essential event in HR. Accumulation of 53BP1 in this context promotes indiscriminate NHEJ and chromosomal instability that ultimately causes proliferation arrest or cell death. Conversely, in cells double deficient in BRCA1 and 53BP1, end-resection is allowed, rescuing HR (Bunting et al., 2010). Consistent with this model, 53BP1 loss reduces the sensitivity of BRCA1-deficient cells to genotoxic agents such as cisplatin and mitomycin C (Bouwman et al., 2010), and to inhibitors of poly-(ADP-ribose) polymerase (PARPi) (Bunting et al., 2010; Farmer et al., 2005), compounds at the forefront for breast cancer therapy (Gartner et al., 2010). Thus, BRCA1-deficient cells are thought to downregulate 53BP1 as a means to ensure proliferation/viability.

Upregulation of 53BP1 levels represents a promising strategy for treatment of breast tumors with the poorest prognosis and for improving their response to PARPi and other DNA damaging strategies. Progress in this regard is hindered by the lack of knowledge about how 53BP1 mRNA and protein levels are downregulated in cancer cells. The inventor previously identified a pathway regulating 53BP1 protein levels (Gonzalez-Suarez et al., 2011; Redwood et al., 2011a; Redwood et al., 2011b). Upregulation of the cysteine protease cathepsin-L (CTSL) leads to accumulation of the protease in the nucleus, degradation of 53BP1 protein, and defects in NHEJ. Importantly, inhibition of CTSL activity by treatment with vitamin D or specific inhibitors stabilizes 53BP1 protein levels and rescues NHEJ defects (Gonzalez-Suarez et al., 2011).

### SUMMARY OF THE INVENTION

Thus, in accordance with the present disclosure, there is provided a method of classifying a subject with triple-negative breast cancer (TNBC) or and BRCA1-related breast cancer as having an active or inactive 53BP1 degradation pathway comprising (a) obtaining a sample from said subject comprising tumor cells; and (b) assessing nuclear protein levels of 53BP1, cathepsin L (CSTL) and vitamin D receptor in said tumor cells, wherein if (i) the patient has high CTSL, low 53BP1 and low VDR, the 53BP1 degradation pathway is active, (ii) the patient has high CTSL, high 53BP1, and high VDR, the 53BP1 degradation pathway is inactive, and (iii) the patient has low CTSL and low 53BP1, the 53BP1 degradation pathway is inactive and the 53BP1 gene contains a mutation or the transcript is destabilized.

Obtaining may comprise taking a sample from said subject, such as a tumor biopsy or a nipple aspirate. The method may further comprise treating said patient with vitamin D and/or a cathepsin inhibitor if the 53BP1 degradation pathway is active, and may even further comprise treating said patient with a PARP inhibitor, and yet even further comprise treating with a cis-platinum agent and/or radiation.

The method may further comprise treating said patient with a PARP inhibitor, a cisplatinum agent and/or radiation if the 53BP1 degradation pathway is inactive. The patient may have previously received a PARP inhibitor, a cisplatinum agent and/or radiation. The cancer may be resistant to a PARP inhibitor, a cisplatinum agent and/or radiation. The cancer may be recurrent or metastatic. The cancer may be TNBC and/or BRCA1-related breast cancer.

The method may further comprise treating the subject if the 53BP1 degradation pathway is inactive and VDR is low. Assessing may comprise immunohistochemistry or ELISA or RIA. The method may further comprise assessing transcripts for CTSL, 53BP1 and/or VDR, such as by RT-PCR. Assessing may further comprise assessing BRCA1 status.

Any embodiment discussed with respect to one aspect of the invention applies to other aspects of the invention as well.

The embodiments in the Examples section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1A-G. Bypass of growth arrest following BRCA1 loss is associated with upregulation of CTSL and degradation of 53BP1. (FIG. 1A) MCF7 cells were lentivirally transduced with a shRNA for depletion of BRCA1 (sh BRCA1) or a control shRNA scrambled (sh scr) and the levels of BRCA1 were assessed by western blot immediately after selection. (FIG. 1B) Proliferation rate of MCF7 cells shows that depletion of BRCA1 induces growth arrest for up to 14 days. (FIG. 1C) Western blots showing 53BP1 and CTSL (Pro-CTSL and Active-CTSL) levels in growth-arrested cells. (FIG. 1D) Proliferation rate of BRCA1-deficient cells that overcome growth arrest (BOGA cells). (FIG. 1E) Western blots showing the levels of BRCA1, 53BP1 and CTSL in control and BOGA cells (representative experiment out of 20 biological repeats). Note how BOGA cells exhibit significantly higher levels of CTSL and lower levels of 53BP1 than control cells. (FIG. 1F) Relative expression of BRCA1, CTSL and 53BP1 in control and BOGA cells as determined by qRT-PCR. Note the increase in transcripts levels of CTSL without significant changes in 53BP1 transcripts levels, indicating destabilization of 53BP1 protein levels. The average±standard deviation of 3 independent experiments is shown. * p value of statistical significance (*p≦0.05). NS, no statistically significant differences. (FIG. 1G) Western blots showing the levels of BRCA1 and 53BP1 upon reconstitution of BRCA1 by transient transfection into BRCA1-deficient cell lines—BOGA cells (left panels) and HCC1937 (right panels). Transfection of an empty vector (EV) was used as control. β-tubulin was used as loading control. Note how reconstitution of BRCA1 in BRCA1-deficient cells stabilizes 53BP1 protein levels. Overall, this figure demonstrates that loss of BRCA1 induces transcriptional upregulation of CTSL and a concomitant destabilization of 53BP1 protein.

FIGS. 2A-E. CTSL is responsible for the degradation of 53BP1 that allows bypass of growth arrest in BOGA cells. (FIG. 2A) MCF7 cells were transduced with shRNAs specific for depletion of 53BP1 (sh 53BP1), BRCA1 (sh BRCA1), or both combined. After selection (puromycin and/or G418), BRCA1, 53BP1, and CTSL levels were monitored by western. (FIG. 2B) Proliferation rate of MCF7 cells deficient in either 53BP1, BRCA1 or both showing that depletion of 53BP1 prevents growth arrest upon BRCA1 loss. This indicates that the decrease in 53BP1 is responsible for the bypass of growth arrest in BOGA cells. (FIG. 2C) Control and BOGA cells were transduced with sh CTSL or sh scr and the levels of CTSL, BRCA1, and 53BP1 monitored by western blot. Note how depletion of CTSL rescues the levels of 53BP1 in BRCA1-deficient cells. Thus, CTSL is responsible for the decrease in 53BP1 levels in BOGA cells. (FIG. 2D) Control or BOGA cells were treated with vitamin D (10⁻⁷M) or vehicle (BGS) and the levels of 53BP1 and p107, a known target of CTSL degradation, were monitored by western blot. Note how inhibition of CTSL by vitamin D stabilizes 53BP1 protein. (FIG. 2E) Control or BOGA cells were incubated with the broad cathepsin inhibitor E-64 (10 μM) or with vehicle (H₂O), and the levels of 53BP1 and CTSL monitored by western blot. Note how inhibition of cathepsin activity stabilizes 53BP1 protein (representative experiment of two biological repeats).

FIGS. 3A-D. Regulation of 53BP1 IRIF in BOGA cells by vitamin D treatment. (FIG. 3A) Immunofluorescence to detect 53BP1 IRIF in control MCF7 cells (sh scr) and growth-arrested BRCA1-depleted cells (sh BRCA1) after 8 Gy of IR. Growth-arrested cells were able to form 53BP1 IRIF, consistent with the normal levels of 53BP1 observed in these cells by western. (FIG. 3B) The same assay was performed as in FIG. 3A, but after bypass of growth arrest (BOGA cells). These cells were unable to form 53BP1 IRIF, consistent with the decreased levels of the protein observed by western. (FIG. 3C) Immunofluorescence studies performed in control and BOGA cells treated with vitamin D or vehicle 24 hr prior to IR. Note how treatment with vitamin D restores the ability of BOGA cells to form 53BP1 IRIF. (FIG. 3D) Graph showing the percentage of cells that respond to radiation by forming 53BP1 IRIF in the presence of vitamin D or vehicle. Cells with >10 53BP1 IRIF were considered positive. At least 1000 cells were analyzed per condition.

FIGS. 4A-D. Regulation of RAD51 IRIF in BOGA cells by CTSL and vitamin D. (FIG. 4A) Immunofluorescence studies of RAD51 IRIF after 8 Gy of IR were performed in control and BOGA cells following treatment with vitamin D or vehicle. (+) denotes positive cells (>10 IRIF) and (−) negative cells. (FIG. 4B) Graph showing the percentage of cells that form RAD51 IRIF 1 h post-IR in the presence of vitamin D or vehicle. Note how BOGA cells are able to form RAD51 foci 1 h post-IR and how vitamin D partially inhibits RAD51 foci formation. This provides an unprecedented role for vitamin D in the regulation of RAD51 foci formation, and thus repair by HR. (FIG. 4C) Quantitation of RAD51 IRIF in control or BOGA cells proficient or deficient in CTSL showing reduced number of positive cells in the CTSL-depleted BOGA cells. (FIG. 4D) Quantitation of percentage of cells positive for RAD51 IRIF at different times post-IR and upon treatment with vitamin D or vehicle. Note how BOGA cells exhibit defects in RAD51 IRIF 3 and 6 hr post-IR. Representative experiments are shown in which at least 400 cells were counted per condition.

FIGS. 5A-E. Effect of CTSL inhibition on DNA repair and genomic stability in BOGA cells. (FIG. 5A) Neutral comet assays performed after 8 Gy of IR show higher olive moments, and thus defects in the fast-phase of DNA repair corresponding to NHEJ in BOGA cells Inhibition of CTSL activity by treatment with vitamin D (10⁻⁷ M) 24 hr pre-IR rescues defects in DNA DSBs repair. This demonstrates that by stabilizing 53BP1, vitamin D can impact the kinetics and choice of mechanism of DNA DSBs repair—NHEJ and HR−. (FIG. 5B) Control and BOGA cells were incubated with vehicle control (C) or vitamin D (VD) for 24 hr prior to irradiation with 2 Gy (IR). Cells collected 24 hr post-IR were analyzed for genomic instability by quantitating the percentage of metaphases presenting with chromosomal aberrations, as shown in the images. N, number of independent experiments (50 metaphases analyzed per condition in each experiment). Note how BOGA cells do not present a profound increase in genomic instability after IR, consistent with previous reports in cells deficient in both BRCA1 and 53BP1. However, stabilization of 53BP1 by vitamin D in the context of BRCA1-deficiency causes a marked increase in genomic instability. These findings provide a novel strategy for inducing genomic instability in specific types of breast cancer cells, i.e., BRCA1-deficient. (FIG. 5C) Control and BOGA cells were incubated with the cathepsin inhibitor E-64 or vehicle control (C) for 24 hr prior to IR, and the extent of genomic instability assessed as in FIG. 5B. Note how stabilization of 53BP1 by E-64 increases significantly the extent of genomic instability after IR in BOGA cells. (FIG. 5D) Graph shows relative numbers of BOGA cells 4 days after treatment with IR, vitamin D, or the combination of both. Treatment protocol was as in FIG. 5B. Note the decrease in cell number after combined treatment with vitamin D and IR compared to untreated cells. (FIG. 5E) Graph shows relative numbers of BOGA cells 4 days after treatment with IR, E-64, or the combination of both. Treatment performed as in FIG. 5C. Note how this treatment hinders the recovery of cells after radiation. All values expressed as mean±SEM. N, number of independent experiments; *p value of statistical significance (*p≦0.05).

FIGS. 6A-C. Chromosomal aberrations in response to IR and PARP inhibitors. (FIG. 6A) BOGA cells transduced with a shRNA control (sh scr) or a shRNA for CTSL were irradiated with 2 Gy and the percentage of cells with aberrant metaphases was quantified 24 hr post-IR. A total of 400 metaphases were analysed (50 per condition). Note how depletion of CTSL, which causes stabilization of 53BP1, increases the degree of genomic instability after IR in BOGA cells, similarly to the effect observed in BOGA cells treated with vitamin D or E-64. (FIG. 6B) MCF7 cells depleted of 53BP1 and BRCA1 (sh 53BP1+sh BRCA1) were treated with vitamin D or vehicle 24 hr prior to IR, and the percentage of metaphases with aberrant chromosomes were quantitated. Note how depletion of 53BP1 prevents the profound increase in genomic instability observed in BOGA cells treated with vitamin D and IR (FIG. 5B). A total of 400 metaphases were analysed (50 per condition). (FIG. 6C) BOGA cells were treated with vitamin D (10⁻⁷ M) 24 hr prior to treatment with the PARPi EB-47 (Pi, 1.2 μg/mL) for an additional 48 hr. Graph shows the percentage of metaphases with chromosomal aberrations. Images show the types of chromosomal aberrations observed. Note the increased genomic instability in cells treated with both PARPi and vitamin D. A total of 400 metaphases were analysed (100 per condition).

FIG. 7. A new signature for subsets of TNBC patients and BRCA1-deficient cells. Immunohistochemical analysis was performed in breast tumor tissue microarrays from 249 patients which included four molecular subtypes: Luminal A, Luminal B, HER2, and Triple-Negative. Representative images of IHC labeling with Ki67, ERα, Her2, CTSL and 53BP1 are shown. Note that while cytoplasmic CTSL is observed in all tumor subtypes, nuclear CTSL is markedly upregulated in a subset of TNBC. In addition, TNBC tumors exhibit a marked decrease in 53BP1.

FIGS. 8A-E. Nuclear 53BP1 expression correlates inversely with nuclear CTSL expression in sporadic human breast cancer. (FIGS. 8A-D) Linear regression analysis between Hscores for nuclear 53BP1 and CTSL in sporadic breast cancer samples with Hscores for CTSL>0. Note the high variability in nuclear 53BP1 Hscores in tumors without nuclear CTSL. The linear regression analysis of the association between nuclear 53BP1 and CTSL depicted in FIG. 8A (Linear regression coefficient (r)=−0.42; p=0.02; coefficient of determination (r2)=6.6%) and FIG. 8B (r=−0.93; p=0.0025; r2=29.2%) include Hscores from both TNBC and no TNBC tumors, while FIGS. 8C (r=−0.255; p=0.294; r2=5.8) and 8D (r=−0.89; p=0.0027; r2=80.2%) depict TNBC only. Note the marked increase in % of the variability in nuclear 53BP1 levels that can be explained by changes in nuclear CTSL parameters of FIGS. 8B and 8D when only tumors with nuclear VDR Hscores<120 are included in the regression analysis. (FIG. 8E) Images of immunohistochemical analysis results in TNBC patients. Two different signatures were observed in these patients (as well as in patients with BRCA1 germline mutations). Top panels show the signature of a few TNBC patients—high nuclear CTSL, 53BP1 and VDR−. In these patients, CTSL-mediated degradation of 53BP1 is inactive. Bottom panels show the most common signature in TNBC patients—high nuclear CTSL, low 53BP1 and low nuclear VDR−. In these patients, CTSL-mediated degradation of 53BP1 is active. These patients are likely to be resistant to PARP inhibitiors and thus would benefit from treatment with vitamin D or cathepsin inhibitors in order to increase sensitivity to PARP inhibitors as well as radiation and putatively other DNA damaging therapeutic strategies.

FIG. 9. Activation of CTSL-mediated degradation of 53BP1 allows BRCA1-deficient cells to overcome genomic instability and growth arrest. Depletion of BRCA1 in breast cancer cells leads to defects in HR, genomic instability and growth arrest. Over time, BRCA1-deficient cells activate CTSL-mediated degradation of 53BP1, which rescues HR defects while inhibiting NHEJ. This allows BRCA1-deficient cells to overcome growth arrest. Inhibition of CTSL activity via treatment with vitamin D or specific inhibitors stabilizes 53BP1 protein levels and induces genomic instability in response to IR and PARPi. Furthermore, upregulation of CTSL-mediated degradation of 53BP1 could be regulated by nuclear VDR.

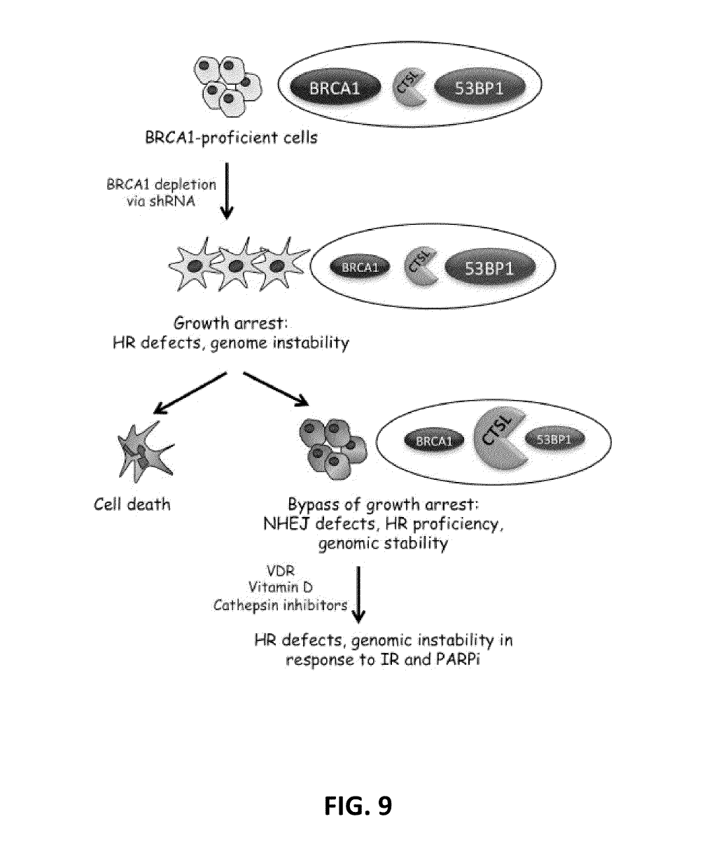

FIGS. 10A-C. Activation of CTSL-mediated degradation of 53BP1 in BRCA1-deficient breast cancer cells. (FIG. 10A) Western blot showing the marked depletion of BRCA1 by one shRNA generated by Dr Junran Zhang. Note the main decrease in signal at 250 kD. Non-specific bands of lower molecular weight are detected by the antibody. (FIG. 10B) MDA-MB-231 breast cancer cells were lentivirally transduced with a shRNA control (sh scr) or the sh BRCA1 as in FIG. 10A. Shortly after selection, BRCA1-deficient cells underwent growth arrest. Approximately 10 days later, MDA-MB-231 cells resumed proliferation. Western blots performed in cells that bypass growth arrest (BOGA cells) show upregulation of CTSL and degradation of 53BP1. (FIG. 10C) MCF7 cells were lentivirally transduced with 5 different shRNAs specific for BRCA1 or a shRNA control (sh scr). Western blots show a reduction of BRCA1 with all hairpins, increased CTSL levels with 4 hairpins, and degradation of 53BP1 with hairpins #3 and #5 (left panels). However, the depletion of BRCA1 and the activation of CTSL-mediated degradation of 53BP1 were modest (left panels). As such, none of the 5 cell lines fully growth arrested. Thus, the inventor combined shRNAs #1 and #2 for depletion of BRCA1. The inventor achieved a marked depletion of BRCA1 and a clear growth arrest. Importantly, BOGA cells generated with these hairpins activated CTSL-mediated degradation of 53BP1 (right panels). Overall, loss of BRCA1 with different shRNAs activates CTSL-mediated degradation of 53BP1 in different types of breast cancer cells.

FIGS. 11A-B. Transcripts levels of CTSL and BRCA1 in cells double deficient in BRCA1/53BP1 or BRCA1/CTSL. (FIG. 11A) MCF7 cells were lentivirally transduced with a shRNA specific for depletion of 53BP1 or a shRNA control (sh scr). After selection, cells were transduced with a shRNA specific for BRCA1. After double selection (puromycin and G418), transcripts levels of CTSL were monitored in the different cell lines. Note how CTSL is upregulated in cells deficient in both 53BP1 and BRCA1 (see FIG. 2A). Average of 2 independent experiments is presented. (FIG. 11B) MCF7 control and BOGA cells were lentivirally transduced with a shRNA specific for depletion of CTSL or a shRNA control. Graphs show the relative expression of CTSL (left graph) and BRCA1 (right graph) in the different cell lines as determined by qRT-PCR. Note the marked downregulation of CTSL transcripts in control and BOGA cells transduced with shCTSL. The average and standard deviation of 3 independent experiments is shown. * p value of statistical significance (*p≦0.05)

FIG. 12. Vitamin D rescues 53BP1 IRIF in BRCA1-deficient cells. BOGA cells were treated with vehicle of vitamin D for 24 hours prior to irradiation with 8 Gy. Cells were fixed 6 hours post-IR and immunofluorescence performed with 53BP1 antibody. More than 1000 cells were counted per condition. The images of large fields show that the effect is not restricted to a few cells, but rather is representative of the whole population of cells.

FIGS. 13A-B. Formation of RAD51 and BRCA1 IRIF in the context of BRCA1-deficiency. (FIG. 13A) MCF7 cells lentivirally transduced with shRNA control (sh scr) or shRNA for depletion of BRCA1 were irradiated with 8 Gy and subjected to immunofluorescence with RAD51 antibody following arrest of BRCA1-deficient cells. Graph shows the quantitation of percentage of cells positive for RAD51 foci formation. Note how growth arrested BRCA1-depleted cells are deficient in RAD51 IRIF. (FIG. 13B) Control and BOGA cells were subjected to immunofluorescence with BRCA1 antibody after irradiation with 8 Gy. Upper panels show the lack of staining in BOGA cells. Graph shows the quantitation of percentage of cells positive for BRCA1 IRIF. Note how BOGA cells are unable to form BRCA1 IRIF. These data demonstrate that BOGA cells remain BRCA1-deficient.

FIGS. 14A-B. Formation of RAD51 IRIF over time in MCF7 control and BOGA cells. (FIG. 14A) Immunofluorescence images showing the formation of RAD51 IRIF in control and BOGA cells at 3 and 6 hours post-irradiation with 8 Gy. Each of the cell lines was treated with vitamin D or vehicle 24 hours prior to irradiation. Note the decrease in RAD51 foci formation at 3 and 6 hours post-IR. (+) denotes positive cells (>10 IRIF) and (−) negative cells. (FIG. 14B) Graph shows quantitation of percentage of control and BOGA cells positive for RAD51 IRIF 1 hour, 3 hours, or 6 hours post-irradiation. Note that while vitamin D impacts on RAD51 foci formation at 1 hour post-IR, it does not exacerbate the defects at later time points. At least 1000 cells were counted per condition.

### DESCRIPTION OF ILLUSTRATIVE EMBODIMENTS

As discussed in detail in the Examples, the inventor has now demonstrated that BRCA1-deficient cells activate CTSL-mediated degradation of 53BP1 as a means to overcome genomic instability and growth arrest. In addition, she finds that depletion or inhibition of CTSL in these cells increases genomic instability in response to ionizing radiation (IR) or PARPi. Lastly, she identifies high levels of nuclear CTSL and low levels of 53BP1 and vitamin D receptor (VDR) as a novel signature in subsets of breast cancer patients.

It is acknowledged that one may find correlations in the literature between loss of 53BP1 and TNBC, loss of VDR and TNBC, and increased CTSL in many types of cancers. However, each biomarker individually does not allow one to identify a specific type of therapy that will address breast cancers having a specific genetic profile, as well as a very aggressive phenotype. For example, 53BP1 has been in the spotlight recently in view of landmark studies demonstrating that 53BP1 is low in TNBC and BRCA1-related tumors. Further, this decrease has been shown to contribute to the resistance of such tumors to PARP inhibitors, drugs at the forefront for breast cancer treatment. Therefore, if a patient is diagnosed with a TNBC and low levels of 53BP1, it is likely that it will be resistant to PARP inhibitors and will not benefit from this type of targeted therapy, unless the levels of 53BP1 can be increased.

In contrast, the triple biomarker signature of the present invention permits one to determine if reduced 53BP1 is due to CTSL-mediated degradation. If that is indeed the case, the combination of vitamin D or cathepsin inhibitors with PARP inhibitors might result in the most beneficial treatment. In contrast, if in this patient the pathway is not active, alternative strategies of treatment will need to be used.

Currently, all TNBC patients are treated in the same manner: surgery, chemotherapy and radiotherapy. Some patients respond while others do not, indicating the need to identify new biomarkers to discriminate patient subsets. The present invention, identifying a functional relationship between three particular biomarkers, is significant for both the diagnosis of breast cancer and for customization of breast cancer treatment. These and other aspects of the invention are described in detail below.

### I. Breast Cancer

A. Background

Breast cancer is a cancer that starts in the breast, usually in the inner lining of the milk ducts or lobules. There are different types of breast cancer, with different stages (spread), aggressiveness, and genetic makeup. With best treatment, 10-year disease-free survival varies from 98% to 10%. Treatment is selected from surgery, drugs (chemotherapy), and radiation. In the United States, there were 216,000 cases of invasive breast cancer and 40,000 deaths in 2004. Worldwide, breast cancer is the second most common type of cancer after lung cancer (10.4% of all cancer incidence, both sexes counted) and the fifth most common cause of cancer death. In 2004, breast cancer caused 519,000 deaths worldwide (7% of cancer deaths; almost 1% of all deaths). Breast cancer is about 100 times as frequent among women as among men, but survival rates are equal in both sexes.

B. Symptoms

The first symptom, or subjective sign, of breast cancer is typically a lump that feels different from the surrounding breast tissue. According to the The Merck Manual, more than 80% of breast cancer cases are discovered when the woman feels a lump. According to the American Cancer Society, the first medical sign, or objective indication of breast cancer as detected by a physician, is discovered by mammogram. Lumps found in lymph nodes located in the armpits can also indicate breast cancer. Indications of breast cancer other than a lump may include changes in breast size or shape, skin dimpling, nipple inversion, or spontaneous single-nipple discharge. Pain (“mastodynia”) is an unreliable tool in determining the presence or absence of breast cancer, but may be indicative of other breast health issues.

When breast cancer cells invade the dermal lymphatics—small lymph vessels in the skin of the breast—its presentation can resemble skin inflammation and thus is known as inflammatory breast cancer (IBC). Symptoms of inflammatory breast cancer include pain, swelling, warmth and redness throughout the breast, as well as an orange-peel texture to the skin referred to as “peau d'orange.” Another reported symptom complex of breast cancer is Paget's disease of the breast. This syndrome presents as eczematoid skin changes such as redness and mild flaking of the nipple skin. As Paget's advances, symptoms may include tingling, itching, increased sensitivity, burning, and pain. There may also be discharge from the nipple. Approximately half of women diagnosed with Paget's also have a lump in the breast.

Occasionally, breast cancer presents as metastatic disease, that is, cancer that has spread beyond the original organ. Metastatic breast cancer will cause symptoms that depend on the location of metastasis. Common sites of metastasis include bone, liver, lung and brain. Unexplained weight loss can occasionally herald an occult breast cancer, as can symptoms of fevers or chills. Bone or joint pains can sometimes be manifestations of metastatic breast cancer, as can jaundice or neurological symptoms. These symptoms are “non-specific,” meaning they can also be manifestations of many other illnesses.

C. Risk Factors

The primary risk factors that have been identified are sex, age, childbearing, hormones, a high-fat diet, alcohol intake, obesity, and environmental factors such as tobacco use, radiation and shiftwork. No etiology is known for 95% of breast cancer cases, while approximately 5% of new breast cancers are attributable to hereditary syndromes. In particular, carriers of the breast cancer susceptibility genes, BRCA1 and BRCA2, are at a 30-40% increased risk for breast and ovarian cancer, depending on in which portion of the protein the mutation occurs. Experts believe that 95% of inherited breast cancer can be traced to one of these two genes. Hereditary breast cancers can take the form of a site-specific hereditary breast cancer—cancers affecting the breast only—or breast-ovarian and other cancer syndromes. Breast cancer can be inherited both from female and male relatives.

D. Subtypes

Breast cancer subtypes are typically categorized on an immunohistochemical basis. Subtype definitions are generally as follows:
normal (ER+, PR+, HER2+, cytokeratin 5/6+, and HER1+)
luminal A (ER+ and/or PR+, HER2−)
luminal B (ER+ and/or PR+, HER2+)
triple-negative (ER−, PR−, HER2−)
HER2+/ER−(ER−, PR−, and HER2+)
unclassified (ER−, PR−, HER2−, cytokeratin 5/6−, and HER1−)
   In the case of triple-negative breast cancer cells, the cancer's growth is not driven by estrogen or progesterone, or by growth signals coming from the HER2 protein. By the same token, such cancer cells do not respond to hormonal therapy, such as tamoxifen or aromatase inhibitors, or therapies that target HER2 receptors, such as Herceptin®. About 10-20% of breast cancers are found to be triple-negative. It is important to identify these types of cancer so that one can avoid costly and toxic effects of therapies that are unlike to succeed, and to focus on treatments that can be used to treat triple-negative breast cancer. Like other forms of breast cancer, triple-negative breast cancer can be treated with surgery, radiation therapy, and/or chemotherapy. One particularly promosing approach is “neoadjuvant” therapy, where chemo- and/or radiotherapy is provided prior to surgery. Another drug therapy is the use of poly (ADP-ribose) polymerase, or PARP inhibitors.

E. Traditional Screening and Diagnosis

While screening techniques discussed above are useful in determining the possibility of cancer, a further testing is necessary to confirm whether a lump detected on screening is cancer, as opposed to a benign alternative such as a simple cyst. In a clinical setting, breast cancer is commonly diagnosed using a “triple test” of clinical breast examination (breast examination by a trained medical practitioner), mammography, and fine needle aspiration cytology. Both mammography and clinical breast exam, also used for screening, can indicate an approximate likelihood that a lump is cancer, and may also identify any other lesions. Fine Needle Aspiration and Cytology (FNAC), performed as an outpatient procedure using local anaesthetic, involves attempting to extract a small portion of fluid from the lump. Clear fluid makes the lump highly unlikely to be cancerous, but bloody fluid may be sent off for inspection under a microscope for cancerous cells. Together, these three tools can be used to diagnose breast cancer with a good degree of accuracy. Other options for biopsy include core biopsy, where a section of the breast lump is removed, and an excisional biopsy, where the entire lump is removed.

Breast cancer screening is an attempt to find cancer in otherwise healthy individuals. The most common screening method for women is a combination of x-ray mammography and clinical breast exam. In women at higher than normal risk, such as those with a strong family history of cancer, additional tools may include genetic testing or breast Magnetic Resonance Imaging.

Breast self-examination was a form of screening that was heavily advocated in the past, but has since fallen into disfavour since several large studies have shown that it does not have a survival benefit for women and often causes considerably anxiety. This is thought to be because cancers that could be detected tended to be at a relatively advanced stage already, whereas other methods push to identify the cancer at an earlier stage where curative treatment is more often possible.

X-ray mammography uses x-rays to examine the breast for any uncharacteristic masses or lumps. Regular mammograms are recommended in several countries in women over a certain age as a screening tool.

Genetic testing for breast cancer typically involves testing for mutations in the BRCA genes. This is not generally a recommended technique except for those at elevated risk for breast cancer.

F. Treatments

The mainstay of breast cancer treatment is surgery when the tumor is localized, with possible adjuvant hormonal therapy (with tamoxifen or an aromatase inhibitor), chemotherapy, and/or radiotherapy. At present, the treatment recommendations after surgery (adjuvant therapy) follow a pattern. Depending on clinical criteria (age, type of cancer, size, metastasis) patients are roughly divided into high risk and low risk cases, with each risk category following different rules for therapy. Treatment possibilities include radiation therapy, chemotherapy, hormone therapy, and immune therapy.

Targeted cancer therapies are treatments that target specific characteristics of cancer cells, such as a protein that allows the cancer cells to grow in a rapid or abnormal way. Targeted therapies are generally less likely than chemotherapy to harm normal, healthy cells. Some targeted therapies are antibodies that work like the antibodies made naturally by one's immune system. These types of targeted therapies are sometimes called immune-targeted therapies.

There are currently 3 targeted therapies doctors use to treat breast cancer. Herceptin® (trastuzumab) works against HER2-positive breast cancers by blocking the ability of the cancer cells to receive chemical signals that tell the cells to grow. Tykerb® (lapatinib) works against HER2-positive breast cancers by blocking certain proteins that can cause uncontrolled cell growth. Avastin® (bevacizumab) works by blocking the growth of new blood vessels that cancer cells depend on to grow and function.

Hormonal (anti-estrogen) therapy works against hormone-receptor-positive breast cancer in two ways: first, by lowering the amount of the hormone estrogen in the body, and second, by blocking the action of estrogen in the body. Most of the estrogen in women's bodies is made by the ovaries. Estrogen makes hormone-receptor-positive breast cancers grow. So reducing the amount of estrogen or blocking its action can help shrink hormone-receptor-positive breast cancers and reduce the risk of hormone-receptor-positive breast cancers coming back (recurring). Hormonal therapy medicines are not effective against hormone-receptor-negative breast cancers.

There are several types of hormonal therapy medicines, including aromatase inhibitors, selective estrogen receptor modulators, and estrogen receptor downregulators. In some cases, the ovaries and fallopian tubes may be surgically removed to treat hormone-receptor-positive breast cancer or as a preventive measure for women at very high risk of breast cancer. The ovaries also may be shut down temporarily using medication.

In planning treatment, doctors can also use PCR tests like Oncotype DX or microarray tests that predict breast cancer recurrence risk based on gene expression. In February 2007, the first breast cancer predictor test won formal approval from the Food and Drug Administration. This is a new gene test to help predict whether women with early-stage breast cancer will relapse in 5 or 10 years, this could help influence how aggressively the initial tumor is treated.

Radiation therapy is also used to help destroy cancer cells that may linger after surgery. Radiation can reduce the risk of recurrence by 50-66% when delivered in the correct dose.

### II. Markers

The present invention utilizes three markers in order to categorize patients for different forms of treatment. These include tumor suppressor p53-binding protein 1 (53BP1), cathepsin L (cathepsin L1; CTSL) and the vitamin D receptor (VDR).

A. 53BP1

53BP1 is a protein that in humans is encoded by the TP53BP1 gene. 53BP1 is underexpressed in most cases of triple-negative breast cancer. In addition to p53, 53BP1 has been shown to interact with Histone H4 dimethylated or monomethylated at Lysine 20, Replication protein A1, RPA2, E2F1, Ataxia telangiectasia mutated, PAXIP1, DYNLL1, H2AFX and Bloom syndrome protein.

In its primary role, 53BP1 binds to the tumor suppressor protein p53 and has a potential role in DNA damage responses. 53BP1 is a key transducer of the DNA damage checkpoint signal and is required for p53 accumulation, G2-M checkpoint arrest, and the intra-S-phase checkpoint in response to ionizing radiation. 53BP1 plays a partially redundant role in phosphorylation of the downstream checkpoint effector proteins BRCA1 and Chk2, but is required for the formation of BRCA1 foci in a hierarchical branched pathway for the recruitment of repair and signaling proteins to sites of DNA damage.

As a checkpoint mediator, 53BP1 co-localizes with phosphorylated H2AX (g-H2AX), Mdc1, the MRN complex, and BRCA1 after treatment with agents that cause DNA DSBs, such as IR and etoposide. 53BP1 has been shown to interact with methylated lysine 20 in histone H4 as well as with methylated lysine 79 of histone H3, and this interaction was inhibited by the suppression of methylation, suggesting that 53BP1 binds directly to histones in response to changes in higher-order chromatin structure that expose methylated lysine residues. Foci formation by both 53BP1 and BRCA1 is dependent on Mdc1 and the RNF8 ubiquitin ligase, although the mechanism by which ubiquitylation affects interactions between the Tudor domain and metylated histones is not understood. 53BP1 has also been reported to have a direct function in DNA DSB repair through stimulation of NHEJ and by mediating long-range interactions between chromosomal ends.

53BP1 also promotes ATM-mediated signalling. siRNA-mediated reduction of 53BP1 levels in human cells reduces the phosphorylation of p53, Chk2, BRCA1, and SMC 1 by ATM, particularly after low doses of damage by ionizing radiation. Thus, 53BP1 may act as a co-activator or mediator of ATM function and its effects on ATM may be enhanced in situations in which the MRN complex is impaired or absent. 53BP1 is also phosphorylated by ATM after DNA damage and this phosphorylation is required for ATM-dependent signalling, although recruitment of 53BP1 to DNA damage sites is independent of its phosphorylation. When 15 conserved SQ/TQ sites in the N-terminus of 53BP lwere mutated, the repair functions of 53BP1 were abrogated, indicating the importance of these modifications. Further, phosphorylation of 53BP1 on serine 25 was required for the binding of the hPTIP protein and for efficient phosphorylation of Chk2 on threonine 68 and BRCA1 on serine 1524.

B. Cathepsin L

Cathepsin L1 is a protein that in humans is encoded by the CTSL1 gene. The protein encoded by this gene is a lysosomal cysteine proteinase that plays a major role in intracellular protein catabolism. Its substrates include collagen and elastin, as well as alpha-1 protease inhibitor, a major controlling element of neutrophil elastase activity. The encoded protein has been implicated in several pathologic processes, including myofibril necrosis in myopathies and in myocardial ischemia, and in the renal tubular response to proteinuria. This protein, which is a member of the peptidase C1 family, is a dimer composed of disulfide-linked heavy and light chains, both produced from a single protein precursor. At least two transcript variants encoding the same protein have been found for this gene. CTSL1 has been shown to interact with Cystatin A.

Studies by the inventor and other investigators have shown that CTSL upregulation leads to accumulation of the protease in the nucleus. In this location, CTSL has been shown to process the transcription factor CDP/CUX, the N-terminal tail of histone H3, as well as 53BP1 and Retinoblastoma family members pRb and p107, but not p130. Importantly, upregulation of oncogenic Ras leads to increased levels of nuclear CTSL, supporting a role for nuclear CTSL in tumorigenesis.

C. Vitamin D Receptor

The vitamin D receptor (VDR) and also known as calcitriol receptor or NR1I1 (nuclear receptor subfamily 1, group I, member 1), is a member of the nuclear receptor family of transcription factors. Upon activation by vitamin D, the VDR forms a heterodimer with the retinoid-X receptor and binds to hormone response elements on DNA resulting in expression or transrepression of specific gene products. In humans, the vitamin D receptor is encoded by the VDR gene. Glucocorticoids are known to decrease expression of VDR, which is expressed in most tissues of the body and regulate intestinal transport of calcium.

This receptor also functions as a receptor for the secondary bile acid lithocholic acid. The receptor belongs to the family of trans-acting transcriptional regulatory factors and shows similarity of sequence to the steroid and thyroid hormone receptors. Mutations in this gene are associated with type II vitamin D-resistant rickets. A single nucleotide polymorphism in the initiation codon results in an alternate translation start site three codons downstream. Alternative splicing results in multiple transcript variants encoding the same protein. The vitamin D receptor plays an important role in regulating the hair cycle. Loss of VDR is associated with hair loss in experimental animals.

Downstream targets of this nuclear hormone receptor are involved principally in mineral metabolism though the receptor regulates a variety of other metabolic pathways, such as those involved in the immune response and cancer. VDR has been shown to interact with BAG1, BAZ1B, CAV3, MED1, MED12, NCOR1, NCOR2, NCOA2, RXRA, RUNX1, RUNX1T1, SNW1, STAT1, and ZBTB16.

D. BRCA-1

BRCA1 (breast cancer 1, early onset) is a human caretaker gene that produces a protein called breast cancer type 1 susceptibility protein, responsible for repairing DNA. The first evidence for the existence of the gene was provided by the King laboratory at UC Berkeley in 1990. Four years later, after an international race to find it, the gene was cloned in 1994 by scientists at Myriad Genetics.

BRCA1 is expressed in the cells of breast and other tissues, where it helps repair damaged DNA, or destroy cells if DNA cannot be repaired. If BRCA1 itself is dysfunctional, damaged DNA is not repaired properly and this increases the risk for cancers.

The protein encoded by the BRCA1 gene combines with other tumor suppressors, DNA damage sensors, and signal transducers to form a large multi-subunit protein complex known as the BRCA1-associated genome surveillance complex (BASC). The BRCA1 protein associates with RNA polymerase II, and through the C-terminal domain, also interacts with histone deacetylase complexes. Thus, this protein plays a role in transcription, DNA repair of double-stranded breaks, ubiquitination, transcriptional regulation, as well as other functions.

The human BRCA1 gene is located on the long (q) arm of chromosome 17 at region 2 band 1, from base pair 38,429,551 to base pair 38,551,283 (Build GRCh37/hg19) (map). BRCA1 orthologs have been identified in most mammals for which complete genome data are available.

The BRCA1 protein contains the following domains:
Zinc finger, C3HC4 type (RING finger)
BRCA1 C Terminus (BRCT) domain
   This protein also contains nuclear localization signal and nuclear export signal motifs. The human BRCA1 protein consists of four major protein domains; the Znf C3HC4-RING domain, the BRCA1 serine domain and two BRCT domains. These domains encode approximately 27% of BRCA1 protein. There are six known isoforms of P38398 BRCA1, with isoforms 1 and 2 comprising 1863 amino acids each.

BRCA1 repairs DNA double-strand breaks. The strands of the DNA double helix are continuously breaking due to endogenous and exogenous damage. Sometimes one strand is broken, and sometimes both strands are broken simultaneously. DNA crosslinking agents are an important source of chromosome/DNA damage. Double strand breaks occur as intermediates after the cross links are removed. BRCA1 is part of a protein complex that repairs DNA when both strands are broken. When both strands are broken, it is difficult for the repair mechanism to “know” how to replace the correct DNA sequence, and there are multiple ways to attempt the repair. The double-stranded repair mechanism that BRCA1 participates in is homologous recombination, in which the repair proteins utilize homologous intact sequence from a sister chromatid, from a homologous chromosome, or from the same chromosome (depending on cell cycle phase) as a template. This DNA repair takes place in the cell nucleus and in the context of chromatin (DNA wrapped around the histone core). Several proteins, including BRCA1, arrive at the histone-DNA complex during repair.

In the nucleus of normal cells, the BRCA1 protein facilitates the recruitment of the protein RAD51 to DNA double-strand breaks. These breaks can be caused by natural radiation or other exposures, but also occur when chromosomes exchange genetic material (homologous recombination). The BRCA2 protein, which has a function similar to that of BRCA1, also interacts with the RAD51 protein. By influencing DNA damage repair, these three proteins play a role in maintaining the stability of the human genome.

BRCA1 directly binds to DNA, with higher affinity for branched DNA structures. This ability to bind to DNA contributes to its ability to inhibit the nuclease activity of the MRN complex as well as the nuclease activity of Mre11 alone. This may explain a role for BRCA1 to promote lower fidelity DNA repair by non-homologous end joining (NHEJ). BRCA1 also colocalizes with γ-H2AX (histone H2AX phosphorylated on serine-139) in DNA double-strand break repair foci, indicating it may play a role in recruiting repair factors.

BRCA1 was shown to co-purify with the human RNA Polymerase II holoenzyme in HeLa extracts, implying it is a component of the holoenzyme. Later research, however, contradicted this assumption, instead showing that the predominant complex including BRCA1 in HeLa cells is a 2 megadalton complex containing SWI/SNF. SWI/SNF is a chromatin remodeling complex. Artificial tethering of BRCA1 to chromatin was shown to decondense heterochromatin, though the SWI/SNF interacting domain was not necessary for this role. BRCA1 interacts with the NELF-B (COBRA1) subunit of the NELF complex. Research suggests that both the BRCA1 and BRCA2 proteins regulate the activity of other genes and play a critical role in embryo development. The BRCA1 protein probably interacts with many other proteins, including tumor suppressors and regulators of the cell division cycle.

Certain variations of the BRCA1 gene lead to an increased risk for breast cancer as part of a hereditary breast-ovarian cancer syndrome. Researchers have identified hundreds of mutations in the BRCA1 gene, many of which are associated with an increased risk of cancer. Women with an abnormal BRCA1 or BRCA2 gene have up to a 60% risk of developing breast cancer by age 90; increased risk of developing ovarian cancer is about 55% for women with BRCA1 mutations and about 25% for women with BRCA2 mutations. These mutations can be changes in one or a small number of DNA base pairs (the building-blocks of DNA). Those mutations can be identified with PCR and DNA sequencing.

In some cases, large segments of DNA are rearranged. Those large segments, also called large rearrangements, can be a deletion or a duplication of one or several exons in the gene. Classical methods for mutations detection (sequencing) are unable to reveal those mutations. Other methods are proposed: Q-PCR, Multiplex Ligation-dependent Probe Amplification (MLPA), and Quantitative Multiplex PCR of Shorts Fluorescents Fragments (QMPSF). New methods have been recently proposed: heteroduplex analysis (HDA) by multi-capillary electrophoresis or also dedicated oligonucleotides array based on comparative genomic hybridization (array-CGH). Some results suggest that hypermethylation of the BRCA1 promoter, which has been reported in some cancers, could be considered as an inactivating mechanism for BRCA1 expression.

A mutated BRCA1 gene usually makes a protein that does not function properly because it is abnormally short. Researchers believe that the defective BRCA1 protein is unable to help fix mutations that occur in other genes. These defects accumulate and may allow cells to grow and divide uncontrollably to form a tumor. BRCA1 mRNA 3′ UTR can be bound by an miRNA, Mir-17 microRNA. It has been suggested that variations in this miRNA along with Mir-30 microRNA could confer susceptibility to breast cancer.

In addition to breast cancer, mutations in the BRCA1 gene also increase the risk of ovarian, fallopian tube, and prostate cancers. Moreover, precancerous lesions (dysplasia) within the Fallopian tube have been linked to BRCA1 gene mutations. Pathogenic mutations anywhere in a model pathway containing BRCA1 and BRCA2 greatly increase risks for a subset of leukemias and lymphomas.

Women having inherited a defective BRCA1 or BRCA2 gene have risks for breast and ovarian cancer that are so high and seem so selective that many mutation carriers choose to have prophylactic surgery. There has been much conjecture to explain such apparently striking tissue specificity. Major determinants of where BRCA1/2 hereditary cancers occur are related to tissue specificity of the cancer pathogen, the agent that causes chronic inflammation or the carcinogen. The target tissue may have receptors for the pathogen, become selectively exposed to an inflammatory process or to a carcinogen. An innate genomic deficit in a tumor suppressor gene impairs normal responses and exacerbates the susceptibility to disease in organ targets. This theory also fits data for several tumor suppressors beyond BRCA1 or BRCA2. A major advantage of this model is that it suggests there may be some options in addition to prophylactic surgery.

### III. Assays for Marker Expression

In some embodiments, the invention involves measuring protein expression in a cell, tissue or subject. Any method of detection known to one of skill in the art falls within the general scope of the present invention. In general, methods of detection focus on nucleic acids (mRNAs) and proteins. Nucleic acids can used be as probes or primers for embodiments involving nucleic acid assessment, whereas antibodies are the most common method for measuring protein expression. Various aspects of target detection as discussed below

A. Protein Based Assays

The present invention will utilize any of a variety of methods to assess protein expression. Samples containing proteins include fluid samples such as blood, serum, sputum, nipple aspirate and ascites. Also, immunohistochemistry utilizes tissue sections which are specially prepared for analysis using antibody probes. The following provides a general discussion of these embodiments.

1. Immunologic Detection

One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (See, e.g., Antibodies: A Laboratory Manual, Cold Spring Harbor Laboratory, 1988; incorporated herein by reference). In particular, antibodies to calcyclin, calpactin I light chain, astrocytic phosphoprotein PEA-15 and tubulin-specific chaperone A are contemplated.

In accordance with the present invention, immunodetection methods are provided. Some immunodetection methods include enzyme linked immunosorbent assay (ELISA), radioimmunoassay (RIA), immunoradiometric assay, fluoroimmunoassay, chemilluminescent assay, bioluminescent assay, and Western blot to mention a few. The steps of various useful immunodetection methods have been described in the scientific literature, such as, e.g., Doolittle & Ben-Zeev (1999); Gulbis & Galand (1993); De Jager et al. (1993); and Nakamura et al. (1987), each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

Immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, immunohistochemistry and the like may also be used.

The antibodies of the present invention may be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1999; Allred et al., 1990).

Also contemplated in the present invention is the use of immunohistochemistry. This approach uses antibodies to detect and quantify antigens in intact tissue samples. Generally, frozen-sections are prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

In an exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies of the invention. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

2. Mass Spectrometry

By exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can generate mass spectrometry profiles that are useful for grading gliomas and predicting glioma patient survival, without regard for the identity of specific proteins. Alternatively, given the established links with calcyclin, calpactin I light chain, astrocytic phosphoprotein PEA-15 and tubulin-specific chaperone A, mass spectrometry may be used to look for the levels of these proteins particularly.

ESI.

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer is delivered to tip through an internal passage from a suitable source (such as from a chromatograph or directly from a sample solution via a liquid flow controller). By applying pressure to the sample in the capillary, the liquid leaves the capillary tip as small, highly electrically-charged droplets, and further undergoes desolvation and breakdown to form single or multicharged gas phase ions in the form of an ion beam. The ions are then collected by the grounded (or negatively charged) interface plate and led through an orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

ESI/MS/MS.

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000).

SIMS.

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

LD-MS and LDLPMS.

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments is due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

MALDI-TOF-MS.

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

B. Nucleic Acid Based Assays

In another aspect, the invention may further utilize detection of mRNA's to determine gene expression. While labile, mRNAs have the advantage of amplification using techniques such as RT-PCR, allowing very small amounts of target to be accurately quantitated. They will also provide complementary information relating to levels that are separate from the issue of protein degradation. The following is a general discussion of such methods.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In particular embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

2. In Situ Hybridization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

3. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific miRNA species isolated from a cell. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of an mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which are incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Chip Technologies and Arrays

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of an miRNA with respect to diagnostic, as well as preventative and treatment methods of the invention.

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### V. Methods of Therapy

In some embodiments, the invention provides compositions and methods for the treatment of breast cancer, including sporadic triple-negative breast cancer (TNBC) and breast cancers from women carrying germline mutations in BRCA1. One of skill in the art will be aware of many treatments that may be combined with the methods of the present invention, some but not all of which are described below. In many contexts, it is not necessary that the tumor cell be killed or induced to undergo normal cell death or “apoptosis,” although such may occur and be beneficial. Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. Patient Subsets

Studies have shown a correlation between global CTSL expression and increased overall degree of malignancy (including breast cancer). Treatment with cathepsin inhibitors had some beneficial effects in some mouse models of tumorigenesis, but not in others. This indicates that monitoring only levels of CTSL does not show predictive value for drug response. Similarly, decreased levels of VDR and vitamin D deficiency are common in TNBC, yet treatment with vitamin D has differential effects in models of breast cancer. Again, merely looking at one marker is insufficient to determine efficacy.

In one aspect, the present invention provides a way to discriminate two distinct patient TNBC subsets by analyzing the three biomarkers discussed above:
Biomarker 1: expression of 53BP1, showing exclusively in the nucleus, which previous studies showed that is low in TN and BRCA1-deficient tumors;
Biomarker 2: expression of nuclear cathepsin L, which has not been previously associated with cancer. Some studies have linked global expression of CTSL with cancer, including breast cancer, but not in human TNBC samples;
Biomarker 3: expression of nuclear VDR, which previous studies showed that is low in TNBC.
   Only with the combination of these three biomarkers is one able to determine if the degradation pathway is active. This is the case if high CTSL, low 53BP1 and low VDR are observed, and it is inactive if high CTSL, high 53BP1 and high VDR are observed.

The significance of this observation is because loss of 53BP1 is prevalent in TNBC and BRCA1-related tumors and associated with resistance to PARP inhibitors and other DNA damaging strategies. However, the causative “agent” was not known, and thus it was also not known how to revert the loss of 53BP1. The present invention has now identified the protease cathepsin L as mediating 53BP1's degradation. Further, an inhibitor of the pathway (VDR) has been identified. Thus, where subsets of patients are identified in which CTSL-mediated degradation of 53BP1 is active, one will treat these patients with vitamin D or cathepsin inhibitors in order to stabilize 53BP1 levels. This treatment in turn is expected to increase sensitivity to PARP inhibitors, and putatively cis-platinum and radiation. Furthermore, although some TNBC patients respond well initially to these treatments, eventually they become resistant. By analyzing those recurrent tumors with the triple biomarker signature, one can determine if the degradation of 53BP1 is activated during recurrence, again providing a possible therapeutic strategy at this late stage in cancer progression for which few other treatment options exist.

Moreover, it is also important to not treat patients where they will be not benefit from the treatment, given both the side effects of such therapy, as well as the high cost. The triple biomarker signature disclosed here will identify patients in which the pathway is off—patients with high CTSL, high 53BP1, and high VDR—and these patients will not benefit from vitamin D treatment or cathepsin inhibitors, and therefore alternative treatments will need to be utilized.

There is yet a third subset of patients in which 53BP1 is lost independently of the levels of CTSL, i.e., patients showing low CTSL and low 53BP1. These patients are likely to have either mutation in the 53BP1 gene, leading to loss of function, or downregulation of the expression of 53BP1 at the level of transcription. These patients might not benefit either from treatment with vitamin D or cathepsin inhibitors.

B. PARP Inhibition, CTSL Inhibition and Vitamin D

As discussed above, only a combination of 3 nuclear biomarkers—53BP1, CTSL and VDR—shows the activation of a pathway in breast cancers having the poorest prognosis, as well as the pathway for therapeutic inhibition. Using the methods disclosed herein, the inventor proposes the clinical categorization of patients with an active degradation pathway (high CTSL) that can benefit from targeted therapy. Further, by monitoring the level of VDR, one can determine whether vitamin D or cathepsin inhibitors should be used. For example, if a patient exhibits high CTSL, low 53BP1, and high VDR, vitamin D may not have much of an effect, since VDR is already high. In this case, inhibition of CTSL activity by specific inhibitors might have a better chance therapeutic effect. In contrast, if a patient exhibits high CTSL, low 53BP1, and low VDR, vitamin D is more likely to benefit this person. In both of these scenarios, following these initial treatments (cathepsin inhibitors/vitamin D), PARP inhibitors may become more effective. Finally, if 53BP1 is not low, PARP inhibition should be effective. Thus, the expression of CTSL, VDR, and 53BP1 could be determined as part of a method for tailoring breast cancer therapy to a specific patient.

1. PARP Inhibition

PARP inhibitors are a group of pharmacological inhibitors of the enzyme poly ADP ribose polymerase (PARP). They are developed for multiple indications; the most important is the treatment of cancer. Several forms of cancer are more dependent on PARP than regular cells, making PARP an attractive target for cancer therapy. In addition to their use in cancer therapy, PARP inhibitors are considered a potential treatment for acute life-threatening diseases, such as stroke and myocardial infarction, as well as for long-term neurodegenerative diseases.

DNA is damaged thousands of times during each cell cycle, and that damage must be repaired. BRCA1, BRCA2 and PALB2 are proteins that are important for the repair of double-strand DNA breaks by the error-free homologous recombinational repair, or HR, pathway. When the gene for either protein is mutated, the change can lead to errors in DNA repair that can eventually cause breast cancer. When subjected to enough damage at one time, the altered gene can cause the death of the cells. PARP1 is a protein that is important for repairing single-strand breaks (‘nicks’ in the DNA). If such nicks persist unrepaired until DNA is replicated (which must precede cell division), then the replication itself can cause double strand breaks to form.

Drugs that inhibit PARP 1 cause multiple double strand breaks to form in this way, and in tumors with BRCA1, BRCA2 or PALB2 mutations these double strand breaks cannot be efficiently repaired, leading to the death of the cells. Normal cells that don't replicate their DNA as often as cancer cells, and that lack any mutated BRCA1 or BRCA2 still have homologous repair operating, which allows them to survive the inhibition of PARP. Some cancer cells that lack the tumor suppressor PTEN may be sensitive to PARP inhibitors because of downregulation of Rad51, a critical homologous recombination component, although other data suggest PTEN may not regulate Rad51. Hence PARP inhibitors may be effective against many PTEN-defective tumors. Cancer cells that are low in oxygen (e.g., in fast growing tumors) are sensitive to PARP inhibitors.

Researchers have recently discovered a significant new mechanism of action for three particular PARP inhibitors currently being tested in clinical trials. Prior to this study, PARP inhibitors were thought to work primarily by blocking PARP enzyme activity, thus preventing the repair of DNA damage and ultimately causing cell death. Now, scientists have established that PARP inhibitors have an additional role in localizing PARP proteins at sites of DNA damage, which has relevance to their anti-tumor activity. The trapped PARP protein—DNA complexes are highly toxic to cells because they block DNA replication. Under normal conditions, PARP1 and PARP2 are released from DNA once the repair process is underway. However, when they are bound to PARP inhibitors, PARP1 and PARP2 become trapped on DNA and are more toxic to cells than the unrepaired single-strand DNA breaks that accumulate in the absence of PARP activity, indicating that PARP inhibitors act as PARP poisons.

The main function of radiotherapy is to produce DNA strand breaks, causing severe DNA damage and leading to cell death. Radiotherapy has the potential to kill 100% of any targeted cells, but the dose required to do so would cause unacceptable side effects to healthy tissue. Radiotherapy therefore can only be given up to a certain level of radiation exposure. Combining radiation therapy with PARP inhibitors offers promise, since the inhibitors would lead to formation of double strand breaks from the single-strand breaks generated by the radiotherapy in tumor tissue with BRCA1/BRCA2 mutations. This combination could therefore lead to either more powerful therapy with the same radiation dose or similarly powerful therapy with a lower radiation dose.

Exemplary PARP inhibitors include Iniparib (BSI 201) for breast cancer and squamous cell lung cancer, Olaparib (AZD-2281) for breast, ovarian and colorectal cancer, Rucaparib (AG014699, PF-01367338) for metastatic breast and ovarian cancer, Veliparib (ABT-888) for metastatic melanoma and breast cancer, CEP 9722 for non-small-cell lung cancer (NSCLC), MK 4827, BMN-673 for advanced hematological malignancies and for advanced or recurrent solid tumors, and 3-aminobenzamide, a prototypical PARP inhibitor.

2. CTSL Inhibition

The cysteine protease, cathepsin L is overexpressed in many cancer cell lines and in breast cancer tissue. It is a lysosomal proteolytic enzyme that is also transported into the cell nucleus where it acts on a few known substrates, including the N-terminal tail of histone H3, the transcription factor CDP/CUX, and as shown by the inventor, the Rb family of tumor suppressors and the DNA repair factor 53BP1. Most of the knowledge about the effect of cathepsin L in cancer relates to the secreted protein. Secreted cathepsin L is involved in the degradation of the extracellular matrix (ECM), the promotion of angiogenesis, and the recruitment and differentiation of stem cells. As a consequence, inhibition of cathepsin L holds great promise to delay tumour growth and metastasis. There is considerable interest in the enzyme as a target for synthesis and application of new potential anticancer agents. Recent progress has been made identifying a series of functionalized benzophenone, thiophene, pyridine, and fluorene thiosemicarbazone derivatives, which are potent inhibitors of cathepsin L with activity in the nanomolar range. In addition, selected compounds were found to inhibit the migration and invasion of prostate and breast cancer cells in preliminary experiments, as well as in a OH mammary carcinoma system that models malignant breast tumour growth delay. In particular, a compound named KGP94 that inhibits specifically cathepsin L showed great promise in this mouse model. In addition, anti-angiogenic effects have been demonstrated with the cathepsin L inhibitor NSITC. This compound inhibited tumor growth in the CAM model of angiogenesis and in nude mouse xenograft models.

3. Vitamin D

Vitamin D belongs to a group of fat-soluble secosteroids responsible for intestinal absorption of calcium and phosphate. In humans, vitamin D is unique because it can be ingested as cholecalciferol (vitamin D₃) or ergocalciferol (vitamin D₂) and because the body can also synthesize it (from cholesterol) when sun exposure is adequate.

Although vitamin D is commonly called a vitamin, it is not actually an essential dietary vitamin in the strict sense, as it can be synthesized in adequate amounts by all mammals exposed to sunlight. An organic chemical compound (or related set of compounds) is only scientifically called a vitamin when it cannot be synthesized in sufficient quantities by an organism, and must be obtained from their diet. However, as with other compounds commonly called vitamins, vitamin D was discovered in an effort to find the dietary substance that was lacking in a disease, namely, rickets, the childhood form of osteomalacia. Additionally, like other compounds called vitamins, in the developed world vitamin D is added to staple foods, such as milk, to avoid disease due to deficiency.

Measures of serum levels reflect endogenous synthesis from exposure to sunlight as well as intake from the diet, and it is believed that synthesis may contribute generally to the maintenance of adequate serum concentrations. The evidence indicates that the synthesis of vitamin D from sun exposure works in a feedback loop that prevents toxicity but, because of uncertainty about the cancer risk from sunlight, no recommendations are issued by the Institute of Medicine, USA, for the amount of sun exposure required to meet vitamin D requirements. Accordingly, the Dietary Reference Intakes for vitamin D assume that no synthesis occurs and that all of a person's vitamin D is from their diet, although that will rarely occur in practice.

In the liver vitamin D is converted to calcidiol, which is also known as calcifediol (INN), 25-hydroxycholecalciferol, or 25-hydroxyvitamin D—abbreviated 25(OH)D; and which is the specific vitamin D metabolite that is measured in serum to determine a person's vitamin D status. Part of the calcidiol is converted by the kidneys to calcitriol, the biologically active form of vitamin D. Calcitriol circulates as a hormone in the blood, regulating the concentration of calcium and phosphate in the bloodstream and promoting the healthy growth and remodeling of bone. Calcidiol is also converted to calcitriol outside of the kidneys for other purposes, such as the proliferation, differentiation and apoptosis of cells; calcitriol also affects neuromuscular function and inflammation.

According to the European Food Safety Authority the Tolerable Upper Intake Levels are:
0-12 months: 25 μg/day (1000 IU)
1-10 years: 50 μg/day (2000 IU)
11-17 years: 100 μg/day (4000 IU)
17+: 250 μg/day (10,000 IU)
Pregnant/lactating women: 250 μg/day (10,000 IU)
   According to the United States Institute of Medicine, the recommended dietary allowances of vitamin D are:
1-70 years of age: 600 IU/day (15 μg/day)
71+ years of age: 800 IU/day (20 μg/day)
Pregnant/lactating: 600 IU/day (15 μg/day)
   The Tolerable Upper Intake Level is defined as “the highest average daily intake of a nutrient that is likely to pose no risk of adverse health effects for nearly all persons in the general population.” Although tolerable upper intake levels are believed to be safe, information on the long-term effects is incomplete and these levels of intake are not recommended:
0-6 months of age: 1,000 IU
6-12 months of age: 1,500 IU
1-3 years of age: 2,500 IU
4-8 years of age: 3,000 IU
9-71+ years of age: 4,000 IU
Pregnant/lactating: 4,000 IU

In healthy adults, sustained intake of more than 1250 micrograms/day (50,000 IU) can produce overt toxicity after several months; those with certain medical conditions such as primary hyperparathyroidism are far more sensitive to vitamin D and develop hypercalcemia in response to any increase in vitamin D nutrition, while maternal hypercalcemia during pregnancy may increase fetal sensitivity to effects of vitamin D and lead to a syndrome of mental retardation and facial deformities. Pregnant or breastfeeding women should consult a doctor before taking a vitamin D supplement.

Vitamin D overdose causes hypercalcemia, and the main symptoms of vitamin D overdose are those of hypercalcemia: anorexia, nausea, and vomiting can occur, frequently followed by polyuria, polydipsia, weakness, insomnia, nervousness, pruritus, and, ultimately, renal failure. Proteinuria, urinary casts, azotemia, and metastatic calcification (especially in the kidneys) may develop. Vitamin D toxicity is treated by discontinuing vitamin D supplementation and restricting calcium intake. Kidney damage may be irreversible. Exposure to sunlight for extended periods of time does not normally cause vitamin D toxicity. Within about 20 minutes of ultraviolet exposure in light-skinned individuals (3-6 times longer for pigmented skin), the concentrations of vitamin D precursors produced in the skin reach an equilibrium, and any further vitamin D that is produced is degraded.

Published cases of toxicity involving hypercalcemia in which the vitamin D dose and the 25-hydroxy-vitamin D levels are known all involve an intake of ?40,000 IU (1000 μg) per day. Recommending supplementation, when those supposedly in need of it are labeled healthy, has proved contentious, and doubt exists concerning long term effects of attaining and maintaining high serum 25(OH)D by supplementation.

Several forms (vitamers) of vitamin D exist (see table). The two major forms are vitamin D₂ or ergocalciferol, and vitamin D₃ or cholecalciferol; vitamin D without a subscript refers to either D₂ or D₃ or both. These are known collectively as calciferol. Vitamin D₂ was chemically characterized in 1932. In 1936, the chemical structure of vitamin D₃ was established and proven to result from the ultraviolet irradiation of 7-dehydrocholesterol.

Chemically, the various forms of vitamin D are secosteroids; i.e., steroids in which one of the bonds in the steroid rings is broken. The structural difference between vitamin D₂ and vitamin D₃ is in their side chains. The side chain of D₂ contains a double bond between carbons 22 and 23, and a methyl group on carbon 24.

Vitamin D₃ (cholecalciferol) is produced by ultraviolet irradiation (UV) of its precursor 7-dehydrocholesterol. This molecule occurs naturally in the skin of animals and in milk. Vitamin D₃ can be made by exposure of the skin to UV, or by exposing milk directly to UV (one commercial method).

Vitamin D₂ is a derivative of ergosterol, a membrane sterol named for the ergot fungus, which is produced by some kinds of phytoplankton, invertebrates, yeasts, and higher fungi such as mushrooms. The vitamin ergocalciferol (D₂) is produced in all of these organisms from ergosterol, in response to UV irradiation. Like all forms of vitamin D, it cannot be produced without UV irradiation. D₂ is not produced by green land plants or vertebrates, because they lack the precursor ergosterol. The biological fate for producing 25(OH)D from vitamin D₂ is expected to be the same as for 25(OH)D₃, although some controversy exists over whether or not D₂ can fully substitute for vitamin D₃ in the human diet.

C. Formulations and Routes for Administration to Patients

In some embodiments, the invention provides a method of treating cancer comprising providing to a patient an effective amount of an agent according to the present invention miRNA. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agent, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

D. Cancer Combination Treatments

In some embodiments, the method further comprises treating a patient with cancer with a conventional cancer treatment. One goal of current cancer research is to find ways to improve the efficacy of chemo- and radiotherapy, such as by combining traditional therapies with other anti-cancer treatments. In the context of the present invention, it is contemplated that this treatment could be, but is not limited to, chemotherapeutic, radiation, a polypeptide inducer of apoptosis or other therapeutic intervention. It also is conceivable that more than one administration of the treatment will be desired.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p9′7), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants, e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the secondary treatment is a gene therapy in which a therapeutic polynucleotide is administered before, after, or at the same time as an miRNA is administered. Delivery of an miRNA in conjunction with a vector encoding one of the following gene products may have a combined anti-hyperproliferative effect on target tissues. A variety of proteins are encompassed within the invention, some of which are described below.

a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zacl, p′73, VHL, MMAC1, DBCCR-1, FCC, rsk-3, p2′7, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, ElA, p300, genes involved in angiogenesis (e.g., VEGF, FGF, thrombospondin, BAI-1, GDAIF, or their receptors) and MCC.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

6. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

E. Dosage

An miRNA can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of antagomir (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of antagomir per kg of bodyweight. The unit dose, for example, can be administered by injection (e.g., intravenous or intramuscular, intrathecally, intratumorally or directly into an organ), inhalation, or a topical application.

Delivery of an miRNA directly to an organ can be at a dosage on the order of about 0.00001 mg to about 3 mg per organ, or particularly about 0.0001-0.001 mg per organ, about 0.03-3.0 mg per organ, about 0.1-3.0 mg per organ or about 0.3-3.0 mg per organ.

Significant modulation of target gene expression may be achieved using nanomolar/submicromolar or picomolar/subnamomolar concentrations of the oligonucleotide, and it is typical to use the lowest concentration possible to achieve the desired resultant increased synthesis, e.g., oligonucleotide concentrations in the 1-100 nM range are contemplated; more particularly, the concentration is in the 1-50 nM, 1-25 nM, 1-10 nM, or picomolar range. In particular embodiments, the contacting step is implemented by contacting the cell with a composition consisting essentially of the oligonucleotide.

In one embodiment, the unit dose is administered once a day, e.g., or less frequently less than or at about every 2, 4, 8 or 30 days. In another embodiment, the unit dose is not administered with a frequency (e.g., not a regular frequency). For example, the unit dose may be administered a single time. Because oligonucleotide agent can persist for several days after administering, in many instances, it is possible to administer the composition with a frequency of less than once per day, or, for some instances, only once for the entire therapeutic regimen.

An miRNA featured in the invention can be administered in a single dose or in multiple doses. Where the administration of the miRNA is by infusion, the infusion can be a single sustained dose or can be delivered by multiple infusions. Injection of the miRNA can be directly into the tissue at or near the site of interest. Multiple injections of can be made into the tissue at or near the site.

In a particular dosage regimen, the miRNA is injected at or near a disease site once a day for seven days, for example, into a tumor, a tumor bed, or tumor vasculature. Where a dosage regimen comprises multiple administrations, it is understood that the effective amount of the miRNA administered to the subject can include the total amount of miRNA administered over the entire dosage regimen. One skilled in the art will appreciate that the exact individual dosages may be adjusted somewhat depending on a variety of factors, including the specific antagomir being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. Variations in these dosage levels can be adjusted using standard empirical routines of optimization, which are well-known in the art. The precise therapeutically effective dosage levels and patterns can be determined by the attending physician in consideration of the above-identified factors.

In one embodiment, a subject is administered an initial dose, and one or more maintenance doses of an miRNA. The maintenance dose or doses are generally lower than the initial dose, e.g., one-half less of the initial dose. The maintenance doses are generally administered no more than once every 5, 10, or 30 days. Further, the treatment regimen may last for a period of time which will vary depending upon the nature of the particular disease, its severity and the overall condition of the patient. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

Certain factors may influence the dosage required to effectively treat a subject, including but not limited to the severity of the disease or disorder, previous treatments, the general health and/or age of the subject, and other diseases present. It will also be appreciated that the effective dosage of the antagomir used for treatment may increase or decrease over the course of a particular treatment. Changes in dosage may result and become apparent from the results of diagnostic assays. For example, the subject can be monitored after administering an antagomir composition. Based on information from the monitoring, an additional amount of the antagomir composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀'s found to be effective in in vitro and in vivo animal models.

### V. Examples

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cells.

Cells maintained in DMEM+10% FBS+antibiotics/antimycotics were transduced with shRNAs and selected in media containing 0.5 mg/mL Geneticin G418 or 2 μg/mL puromycin. Except for one shBRCA1 (sequence: TCAGTACAATTAGGTGGGCTT (SEQ ID NO: 1)) that was constructed by Junran Zhang, the rest were purchased from the Genome Institute at Washington University (sh BRCA sequences #1: GAGAATCCTAGAGATACTGAA (SEQ ID NO: 2), #2: TATAGCTGTTGGAAGGACTAG (SEQ ID NO: 3), #3: CCCTAAGTTTACTTCTCTAAA (SEQ ID NO: 4), #4: GCCCACCTAATTGTACTGAAT (SEQ ID NO: 5), #5: CCCACCTAATTGTACTGAATT (SEQ ID NO: 6).

Transductions. Lentiviral transductions were performed as previously described (Gonzalez-Suarez et al., 2009). HCC1937 cells were a gift from Junran Zhang. HA-BRCA1 (Plasmid 14999, Addgene) transient transfections were carried out using the X-tremeGENE HP transfection reagent (06366236001, Roche). MCF7 cells transduced with sh scr or sh BRCA1 were fingerprinted and shown to correspond to parental MCF7 cells (data not shown).

Treatments.

Cells were subjected to the following treatments:

### Vitamin D.

Cells were incubated with 10-7 M 1α,25-dihydroxyvitamin D3 (D1530, Sigma-Aldrich) for 24-48 hours, as indicated. Aliquots of 1 nmol 1α,25-dihydroxyvitamin D3 were resuspended in 1 mL of BGS and diluted in DMEM to a final concentration of 10% BGS. BGS was the vehicle in control media.

### E-64.

Cells were incubated with the broad spectrum cathepsin inhibitor E-64 (E3132, Sigma-Aldrich) diluted in water at a concentration of 10 μM for 24 hours.

### PARPi.

Cells were treated with the PARPi EB-47 (E8405, Sigma-Aldrich) diluted in water at a concentration of 1.2 μg/mL for 48 hours.

### Ionizing Radiation.

For determining the extent of genomic instability, cells were irradiated with 2 Gy and analysis of metaphase spreads was performed 24 hours post-irradiation. For assaying formation of IRIF, cells were irradiated with 8 Gy and fixed and processed for immunofluorescence 1 hour, 3 hours, or 6 hours post-irradiation as indicated. For comet assays, cells were irradiated with 8 Gy.

Assays.

For immunoblotting, immunofluorescence, and comet assays, the inventor followed protocols described previously (Gonzalez-Suarez et al., 2011; Redwood et al., 2011b), except for BRCA1 IF, which was performed following a protocol from Fernandez-Capetillo's laboratory. Briefly, cells were washed twice with PBS, and incubated in CSK I buffer (10 mM PIPES pH6.8, 100 mM NaCl, 300 mM sucrose, 3 mM MgCl2, 1 mM EGTA, and 0.5% Triton X-100) for 5 minutes. Coverslips were washed 5 times with cold PBS and fixed in modified STF buffer (150 mM 2-Bromo-2-nitro-1,3,-propanediol, 108 mM diazolidinyl urea, 10 mM Na citrate, and 50 nM EDTA pH 5.7) for 30 minutes at room temperature. Coverslips were then washed twice with cold PBS and permeabilized (PB buffer: 100 mM Tris-HCl pH 7.4, 50 mM EDTA pH 8.0, and 0.5% Triton X-100) for 15 minutes at room temperature followed by 2 washes in PBS. Blocking and antibody staining were carried out as previously described, with BRCA1 antibody (sc-6954, Santa Cruz) diluted at 1:200 and secondary antibody diluted at 1:1000.

Proliferation Assay.

To quantify growth upon depletion of BRCA1, cells were plated in triplicate at 150,000 cells/well and counted 96 hours later. Cell proliferation was measured in each cell line 4 times within a 14 day period. To quantify the growth of the sh 53BP1/sh BRCA1 cells, 4×106 cells were plated in 15 cm dishes and counted every 96 hours for 20 days. In order to extrapolate proliferation to the respective time periods, the inventor used the equation N=N0ekt where N is the final number of cells, N0 is the starting number of cells, k is ln 2/DT (doubling time), and t is time in days (Sherley et al, 1995). For each 96 hour period the inventor calculated the doubling time and used it to estimate the number of cells (N) that would result from initially plating 150,000 (N0) and culture them for a given period of time. The doubling times for the control cells remained constant throughout the time period, while the doubling times for the shBRCA1 cells started increasing once the cells overcame the growth arrest.

Quantitative Reverse-Transcription PCR.

RNA was isolated using the RNAqueous-4PCR Kit (Ambion). cDNA was generated from 500 ng of total RNA using TaqMan Reverse Transcription Reagents (Applied Biosystems). BRCA1, 53BP1, cathepsin L and 18S expression was determined using TaqMan Gene Expression Assays (Hs01556193_m1, Hs00996818_m1, Hs00377632_m1, Hs99999901_s1, respectively, Applied Biosystems). For the analysis, all reactions (in triplicate) were carried out by amplifying target gene and endogenous controls in the same plate. Relative quantitative evaluation of target gene was determined by comparing the cycle thresholds.

Analysis of Aberrant Chromosomes.

Cells were treated with vitamin D or E-64 for 24 hours, irradiated, and allowed to recover for 24 hours. Cells treated with PARPi were pre-treated with vitamin D for 24 hours followed by combined treatment with vitamin D and PARPi for 48 hours. Following all treatments, cells were arrested in mitosis by treatment with colcemid for 4 hours and metaphase spreads prepared by hypotonic swelling in 0.56% KCl, followed by fixation in 3:1 methanol: acetic acid. Cell suspensions were dropped onto slides and stained for 25 minutes in Wright-Giemsa Stain (9380-32, Ricca Chemical Company) and then washed in water. Slides were allowed to dry and were mounted using Eukitt Mounting Reagent and analyzed on an Olympus BX51 light microscope.

Tissue Tumor Microarrays (TMA).

A total of 249 tissue samples from patients with sporadic breast carcinoma were obtained at Hospital Universitari Arnau de Vilanova in Lleida, Spain from 1998 to 2012. An informed consent was obtained from each patient and the study was approved by the local Ethical Committee. The series of 249 tumor samples included formalin-fixed, paraffin-embedded blocks for all patients, 165 core biopsies, before the initiation of neoadjuvant treatment, and 84 surgical specimens, before the initiation of adjuvant treatment. Tumors were classified according to the expression of the following proteins: Ki67, ERα and Her2 into four molecular subtypes: Luminal A (n=99), Luminal B (n=69), Her2 (n=45), Triple-Negative (n=36). Luminal A tumors are steroid hormone receptor—positive, negative for Her2, contain less than 30% of Ki67 positive cells, and tend to have a good prognosis. Luminal B tumors are steroid hormone receptor—positive, negative for Her2, contain more than 30% Ki67 positive cells, and tend to have a worse prognosis than luminal A. In contrast, Her2 tumors are positive for Her2 and have been shown to have a poor prognosis. Triple-Negative tumors are negative for steroid hormone receptors and Her2 and have the worst prognosis. Moreover, tissue samples were also obtained from 18 breast cancers from patients carrying a BRCA1 germline mutation (Luminal A, n=1; Luminal B, n=1; Her2, n=2; Triple-Negative, n=15), and from 14 breast cancers from patients carrying a BRCA2 germ-line mutation (Luminal A, n=6; Luminal B, n=2; Her2, n=3; Triple-Negative, n=3). These patients had been treated in Hospital Santa Creu I Sant Pau, Barcelona, Spain. A Tissue arrayer device (Beecher Instrument, MD) was used to construct the TMA. Briefly, all samples were histologically reviewed and representative areas were marked in the corresponding paraffin-blocks. Two selected cylinders (0.6 mm of largest diameter) from two different areas were included for each case.

Immunohistochemical Analysis.

TMA blocks were sectioned at a thickness of 3 μm, dried for 1 h at 65° C. before being dewaxed in xylene and rehydrated through descending concentrations of ethanol, and washed with phosphate-buffered saline. Ki67, ERα and HER2 were used to determine molecular subtype. Comparative studies of CTSL, VDR, and 53BP1 expression were carried out on sequential serial sections. Antigen retrieval for CTS L and ERα was achieved by heat treatment at 95° C. for 20 min in a high pH solution (DAKO). Heat-induced antigen retrieval for 53BP1 and Ki67 was performed in a low pH solution (DAKO). Before staining the sections, endogenous peroxidase was blocked. Primary antibodies and incubation times were as follows: CTSL (1:50; S-20, Santa Cruz Biotechnology, incubation overnight at 4° C.); 53BP1 (1:2500; NB100-304, Novus Biologicals, incubation 20 min at room temperature); VDR (1:2000; ABCAM, incubation 20 min at room temperature); Ki67 (Ready-to-use; M1B, DAKO, incubation 20 min at room temperature); ERα(Ready-to-use; 1D5, DAKO, incubation 20 minutes at room temperature), and HER2 (Herceptest Kit, DAKO). The reaction was visualized with the Streptovidin-Biotin Complex (DAKO) for CTSL and Envision Flex (DAKO) for 53BP1, Ki67 and ERα. Sections were counterstained with haematoxylin. Appropriate positive and negative controls were also tested.

Immunohistochemical scores provide a semiquantitative measurement of protein expression per tumor by taking into consideration the percentage of positive cells and the intensity of their staining A histological score ranging from 0 (no immune reaction) to 300 (maximal immunoreactivity) was obtained with the formula Histoscore (Hscore)=1×(% light staining)+2×(% moderate staining)+3×(% strong staining) The reliability of such scores for the interpretation of immunohistochemical staining in TMAs has been reported (Pallares et al., 2009). Her2 staining was evaluated according to a standard protocol (HercepTest; DAKO) and scored as 4 intensities (i.e., negative=0; weak=1+; moderate=2+; and strong=3+), considering negative Her2 expression for intensity values of 0, 1+ and 2+ when there was no amplification by FISH, and positive for intensity values of 3+ and 2+, when 2+ was amplified by FISH. For each marker, there were a variable number of non-assessable cases due to technical problems including no representative tumor sample left in the cylinders, detachment, cylinders missed while constructing the array, necrosis, and absence of viable tumor cells in the TMA sections.

Statistical Analysis.

For the in vitro experiments, a “two-tailed” student's t-test was used to calculate statistical significance of the observed differences. Microsoft Excel v.2010 was used for the calculations. In all cases, differences were considered statistically significant when p<0.05. For the TMA studies, a Kruskal-Wallis test was used to test the statistical significance of the observed differences in CTSL, VDR and 53BP1 Hscores between molecular breast tumor subtypes. Tumors were partitioned according to cut off nuclear Hscores for CTSL, 53BP1 and VDR selected by their median values as >0, <150 and <120, respectively. Once cut off points were applied, Fisher Exact Test was used to assess the statistical significance of the differences in the distribution of the two categories of CTSL, 53BP1 and VDR Hscores above or below cut off points for all breast tumor types. In the analysis of BRCA1 and BRCA2 samples, the inventor used the Mann-Whitney test to analyze Hscore differences between them as well as differences of each of them with the sample of sporadic tumors, and the inventor used the Fisher exact test to assess differences in the distributions of groups defined by the same cut-off points used for the sporadic tumors. The subsample of TNBC was also compared with the sample of germinal BRCA1 mutated cancers using the same statistical tests. The Pearson correlation coefficient together with linear regression models assessed the statistical significance of the relationship between nuclear CTSL and 53BP1 Hscores. R package was used to all TMA statistical tests. Differences were considered significant when p<0.05.

Abbreviations List.

TNBC-Triple negative breast cancer; CTSL-cathepsin L; DSBs-DNA double strand breaks; HRhomologous recombination; NHEJ-non homologous end joining; PARPi-poly-(ADP-ribose) polymerase inhibitor; VDR-vitamin D receptor; IR-ionizing radiation; IRIF-ionizing radiation induced foci; BOGA-BRCA1 deficient cells that overcome growth arrest; IHC-immunohistochemistry.

### Example 2

### Results

BRCA1-Deficient Cells Activate CTSL-Mediated Degradation of 53BP1 to Bypass Growth Arrest.

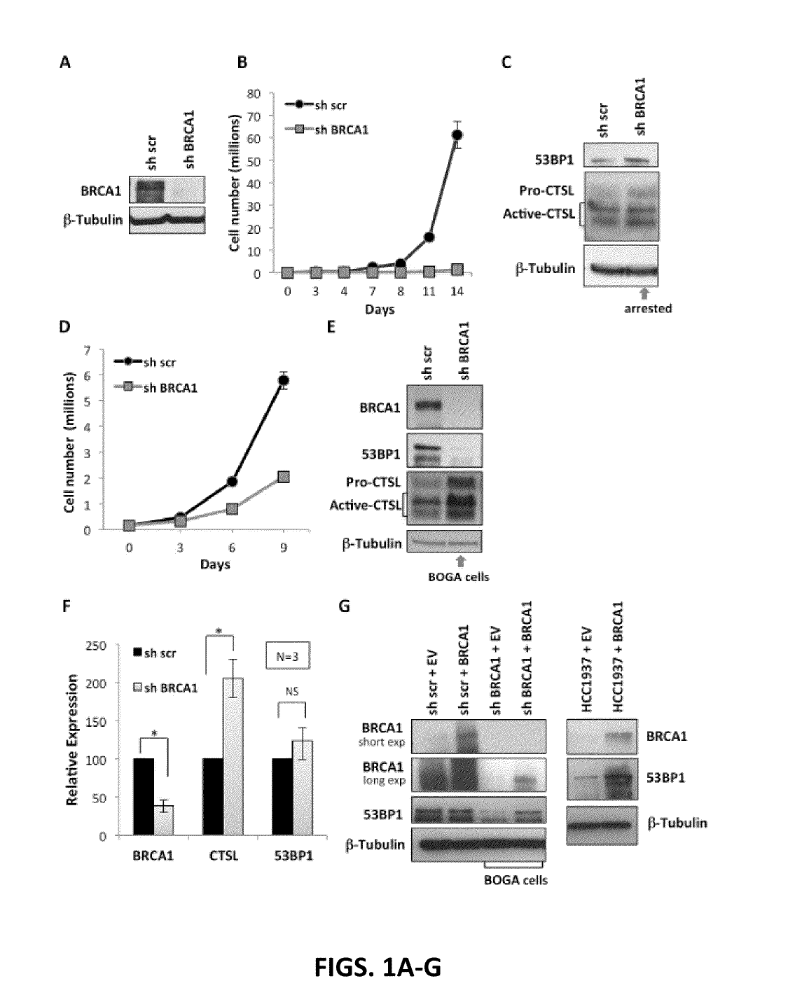

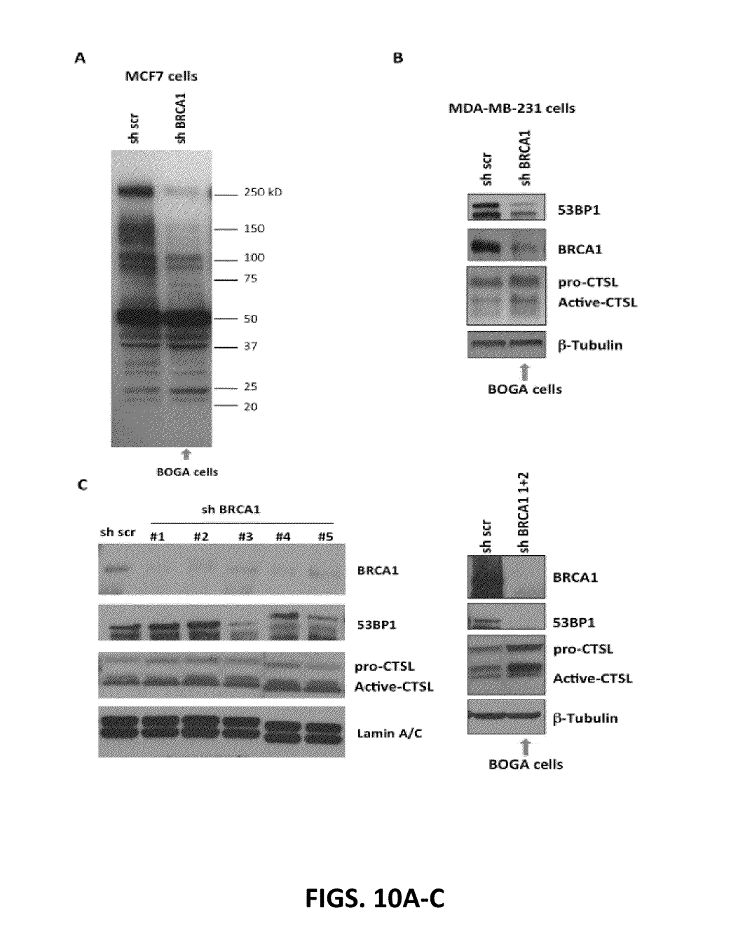

Previous studies demonstrated that loss of 53BP1 rescues the BRCA1 deficient phenotype (Bouwman et al., 2010; Bunting et al., 2010; Cao et al., 2009). The inventor also showed that CTSL regulates the stability of 53BP1 (Gonzalez-Suarez et al., 2011; Redwood et al., 2011a; Redwood et al., 2011b). Here, the inventor investigated whether breast tumor cells are able to downregulate 53BP1 upon loss of BRCA1 in order to restore proliferation/viability and if CTSL is one of the factors responsible for the depletion of 53BP1. The human breast cancer cell line MCF7, which is BRCA1 and 53BP1 proficient, was depleted of BRCA1 via lentiviral transduction with shRNAs (FIG. 1A and FIG. 10A). As previously shown in human fibroblasts (Tu et al., 2011), efficient depletion of BRCA1 in MCF7 cells induces growth arrest (FIG. 1B). Moreover, growth arrested BRCA1-deficient cells did not show differences in the levels of 53BP1 or CTSL proteins when compared to control cells (FIG. 1C).

Interestingly, after approximately two weeks in culture, BRCA1-deficient cells resumed proliferation, albeit at a slower rate than control cells (FIG. 1D). Importantly, BRCA1-deficient cells that Overcome Growth Arrest (herein referred to as BOGA cells, for clarity) exhibit decreased 53BP1 protein levels and increased CTSL levels (FIG. 1E). This signature was also observed in MDA-MB-231 breast cancer cells that overcome the growth arrest induced by depletion of BRCA1 (FIG. 10B). Similar changes in CTSL and 53BP1 protein levels were observed with different shRNAs for depletion of BRCA1 (FIG. 10C). Monitoring of transcripts levels by qRT-PCR revealed that BOGA cells exhibit transcriptional upregulation of CTSL, without significant changes in 53BP1 transcripts levels, indicating a decrease in 53BP1 protein stability (FIG. 1F). To confirm a role for BRCA1 in the regulation of 53BP1 levels, the inventor reconstituted BRCA1 via transient transfection in BOGA cells and the BRCA1-deficient breast cancer cell line HCC1937. As shown in FIG. 1G, ectopic expression of BRCA1 results in stabilization of 53BP1 in both cell lines, revealing a novel function of BRCA1 in the stabilization of 53BP1 protein.

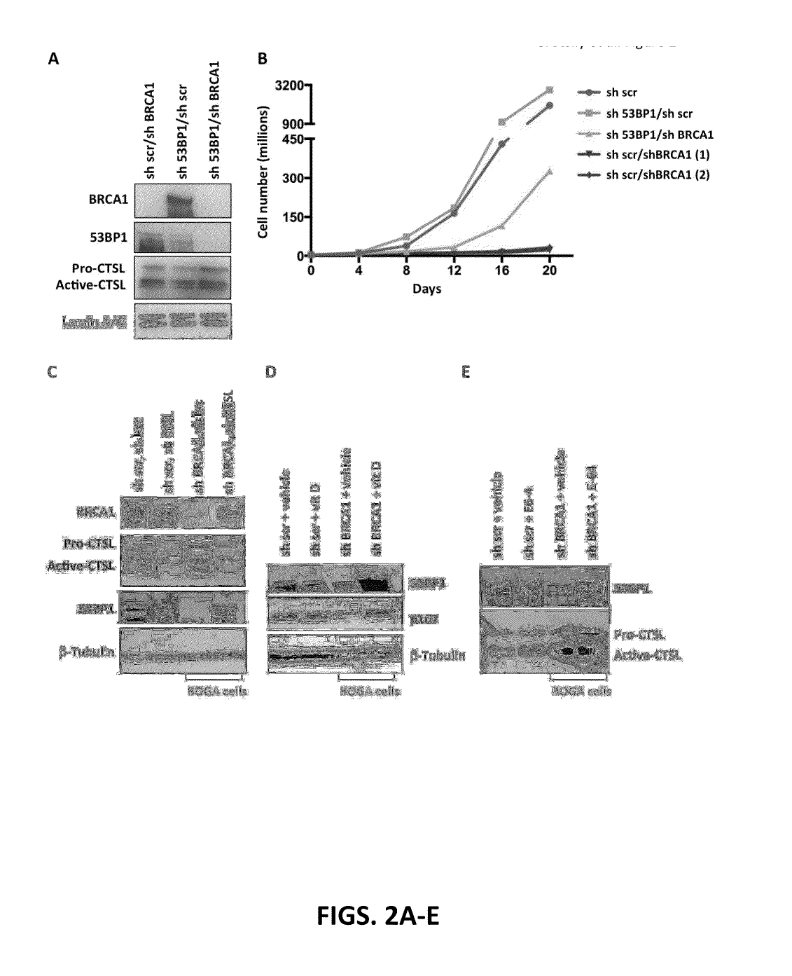

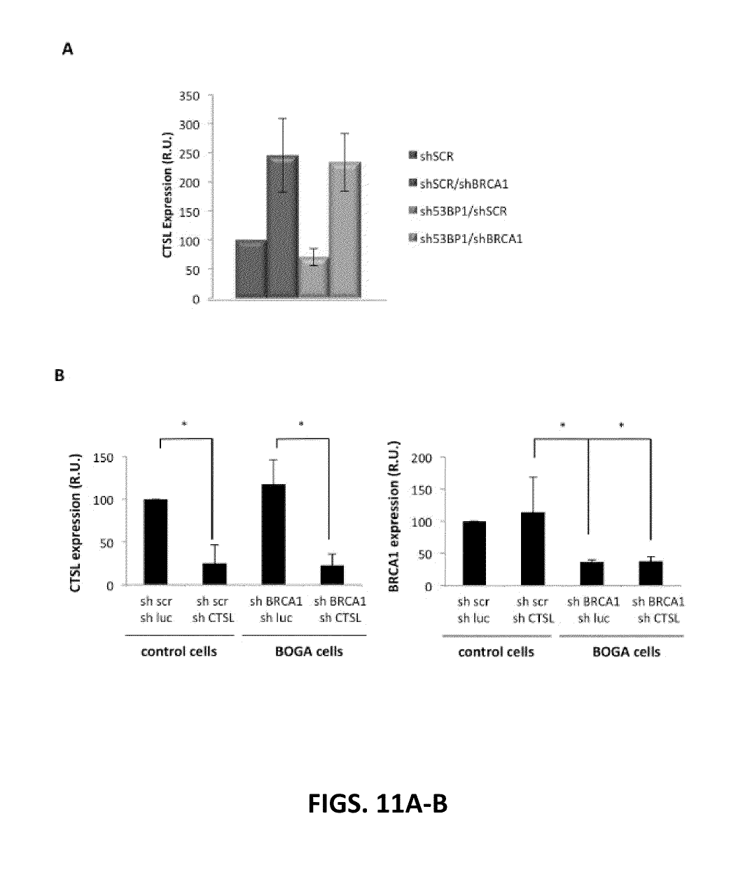

To determine if loss of 53BP1 is responsible for the bypass of growth arrest in BRCA1-deficient cells, the inventor depleted 53BP1 prior to depletion of BRCA1. As shown in FIG. 2A, the inventor achieved a marked reduction in both 53BP1 and BRCA1 protein levels. Importantly, previous depletion of 53BP1 prevented the characteristic growth arrest that follows BRCA1 depletion, as cells continued proliferating, although at a lower rate (FIG. 2B). These data indicate that loss of 53BP1 allows BRCA1-deficient cells to bypass growth arrest. Interestingly, cells that were depleted of 53BP1 prior to depletion of BRCA1 also upregulated CTSL (FIG. 11A), suggesting that upregulation of CTSL is independent of 53BP1 status.

Next, the inventor determined whether CTSL is responsible for the degradation of 53BP1 in BRCA1-deficient cells by performing depletion of CTSL in control and BOGA cells (FIG. 2C and FIG. 11B). Importantly, depletion of CTSL stabilized 53BP1 protein levels in BOGA cells mirroring those of control cells, while transcripts levels of BRCA1 were not affected by depletion of CTSL (FIG. 11B). Intriguingly, the inventor observed a slight increase in BRCA1 protein in BOGA cells depleted of CTSL, suggesting a possible feedback mechanism of CTSL on BRCA1 protein levels, which remains to be tested.

The inventor previously demonstrated that vitamin D inhibits CTSL activity and stabilizes 53BP1 protein in MEFs (Gonzalez-Suarez et al., 2011). Here, the inventor shows that treatment of BOGA cells with vitamin D (1α,25-dihydroxyvitamin D3) stabilizes the levels of 53BP1 (FIG. 2E). Similarly, treatment with the cathepsin inhibitor E-64 leads to increased levels of 53BP1 protein (FIG. 2F). Overall, these data demonstrate that cells growth arrested following BRCA1 loss activate CTSL-mediated degradation of 53BP1 in order to bypass the growth arrest imposed by BRCA1 deficiency. In addition, depletion or inhibition of CTSL can increase 53BP1 levels in the context of BRCA1 deficiency with potential therapeutic effects.

CTSL-Mediated Degradation of 53BP1 Rescues HR Defects in BRCA1-Deficient Cells.

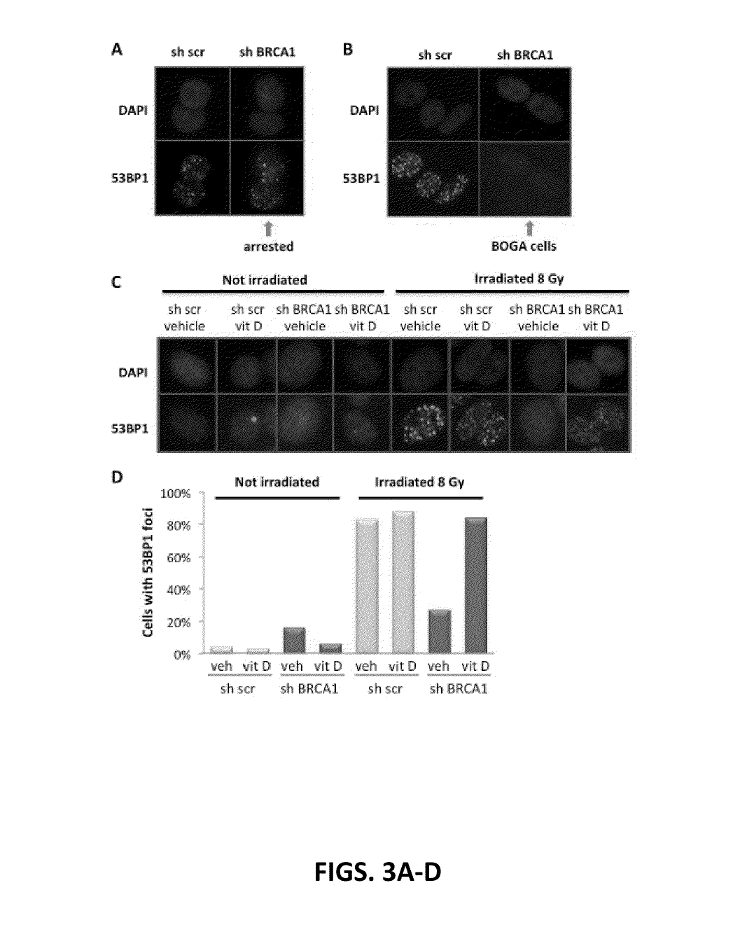

BRCA1 deficiency impairs DNA end-resection at DSBs and formation of RAD51-coated filaments that facilitate subsequent HR steps (Bhattacharyya et al., 2000; Moynahan et al., 1999; Schlegel et al., 2006; Scully et al., 1997a; Scully et al., 1997b; Snouwaert et al., 1999; Sung et al., 2003). Interestingly, loss of 53BP1 in BRCA1-deficient cells partially rescues HR and accumulation of RAD51 at ionizing radiation-induced foci (IRIF) (Bunting et al., 2010). Here, the inventor determined how CTSL-mediated degradation of 53BP1 impacts on 53BP1 and RAD51 recruitment to DNA DSBs. The inventor showed that growth arrested MCF7 cells following BRCA1 depletion retained their ability to recruit 53BP1 protein to IRIF (FIG. 3A), consistent with their normal levels of 53BP1 (FIG. 1C). In contrast, BOGA cells were unable to form 53BP1 IRIF (FIG. 3B), consistent with their decreased 53BP1 levels (FIG. 1E).

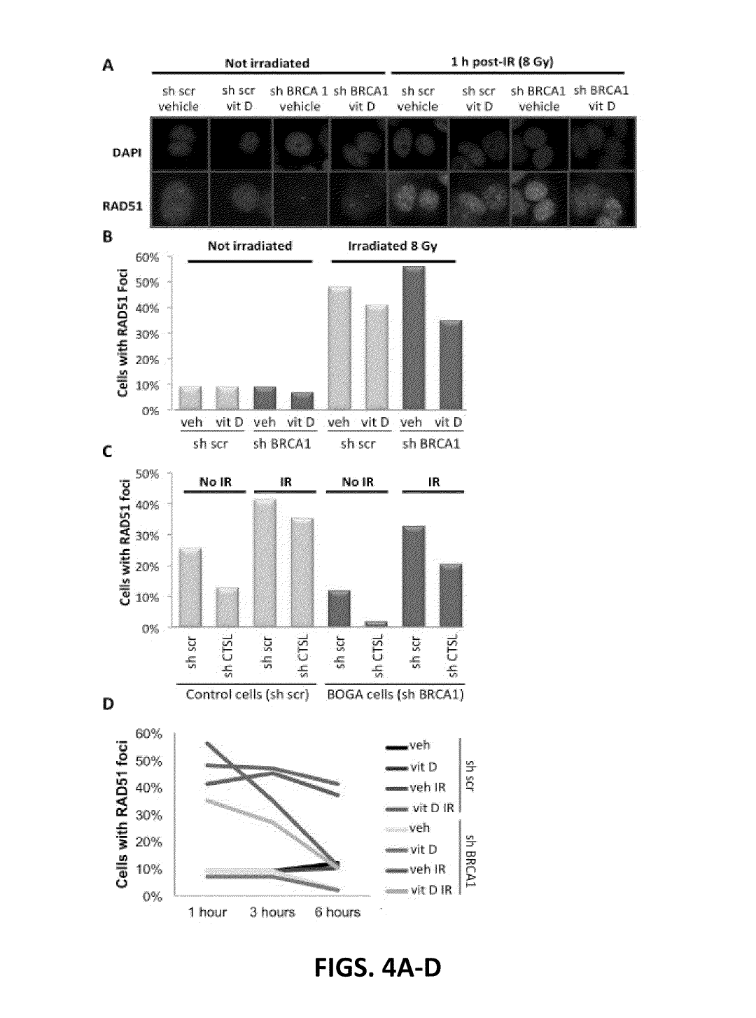

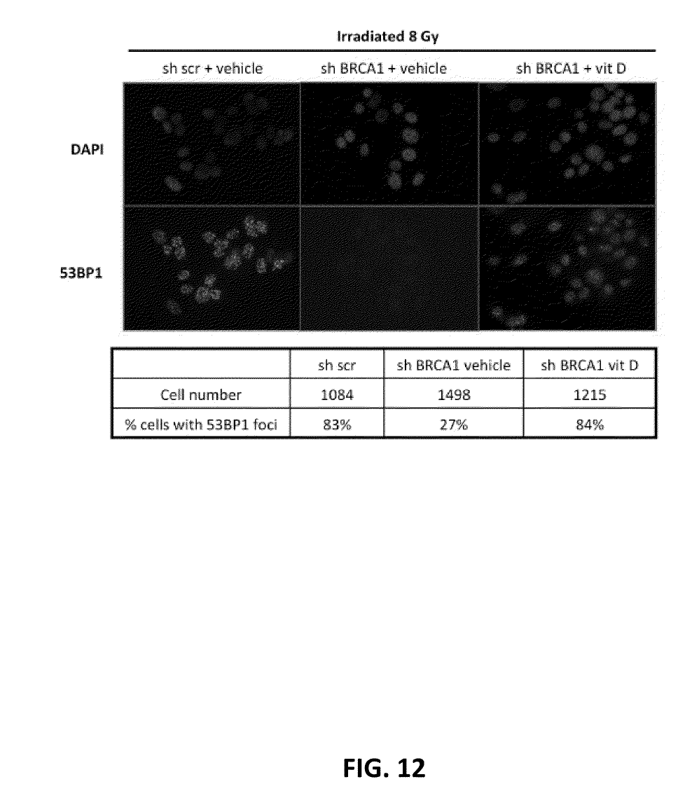

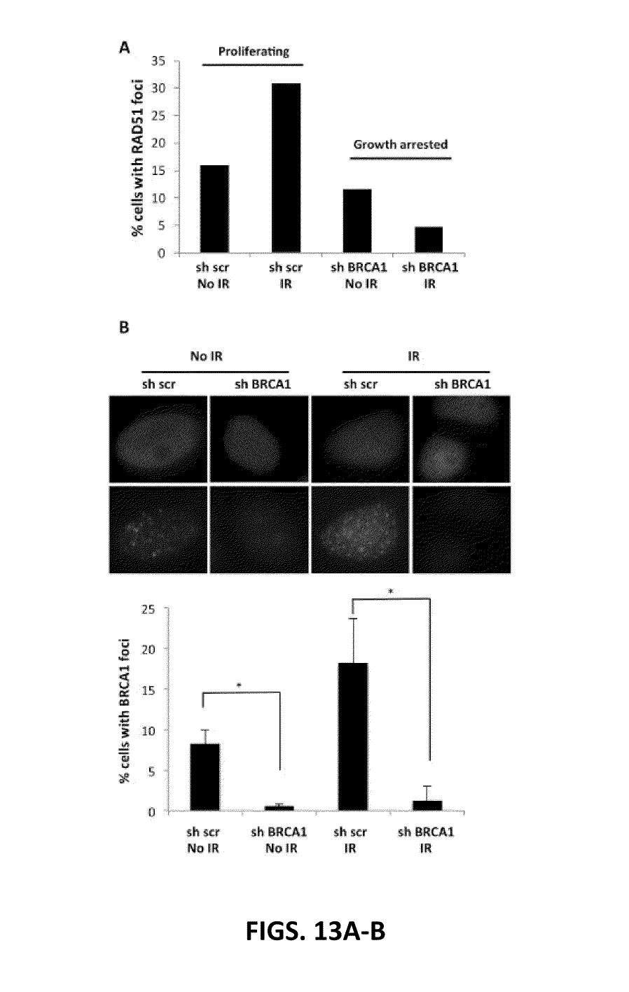

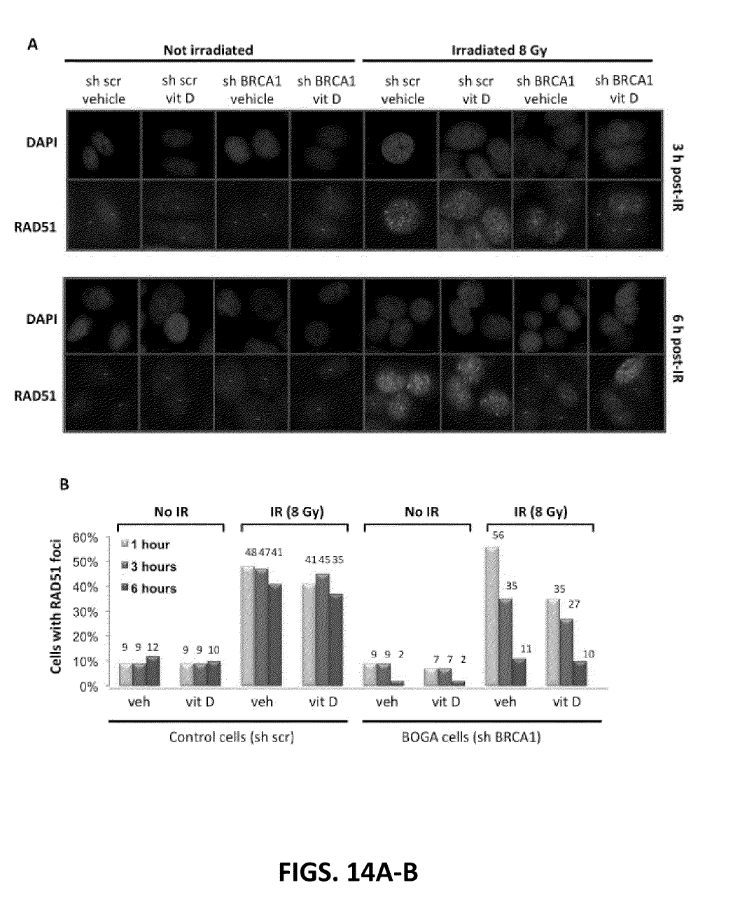

Next, the inventor determined if the deficiency in 53BP1 foci formation could be rescued by inhibiting CTSL. BOGA cells treated with vehicle are defective in the formation of 53BP1 IRIF, while treatment with vitamin D rescued 53BP1 IRIF (FIGS. 3C-D and FIG. 12). In contrast to 53BP1, RAD51 recruitment to IRIF was inhibited shortly after BRCA1 depletion, in growth-arrested cells (FIG. 13A). This defect was rescued in BOGA cells at 1 hour post-IR (FIGS. 4A-B). The rescue was not due to an increase in BRCA1 levels, as BOGA cells were unable to form BRCA1-labeled IRIF (FIG. 13B). Next, the inventor tested whether activation of CTSL-mediated degradation of 53BP1 is behind the rescued recruitment of RAD51 to DSBs in BRCA1-deficient cells. First, the inventor show that stabilization of 53BP1 in BOGA cells by vitamin D reduces the extent of RAD51 IRIF (FIGS. 4A-B), revealing an unprecedented role for vitamin D in modulating HR. Similarly, depletion of CTSL reduces the ability of BOGA cells to recruit RAD51 to DSBs (FIG. 4C). Intriguingly, while control cells form RAD51 IRIF for up to 6 hours, the percentage of BOGA cells positive for RAD51 IRIF decreased significantly over time (FIG. 4D and FIG. 14), indicating that 53BP1 deficiency in BOGA cells does not completely compensate for the absence of BRCA1. These studies demonstrate that by activating CTSL-mediated degradation of 53BP1, BRCA1-deficient cells can rescue to certain extent the HR defects to promote survival, and that this pathway can be disrupted by inhibiting CTSL activity. These findings provide a novel strategy to modulate HR efficiency in BRCA1 deficient cells to induce genomic instability.

Consequences of CTSL-Mediated Degradation of 53BP1 for DNA Repair and Genomic Stability.

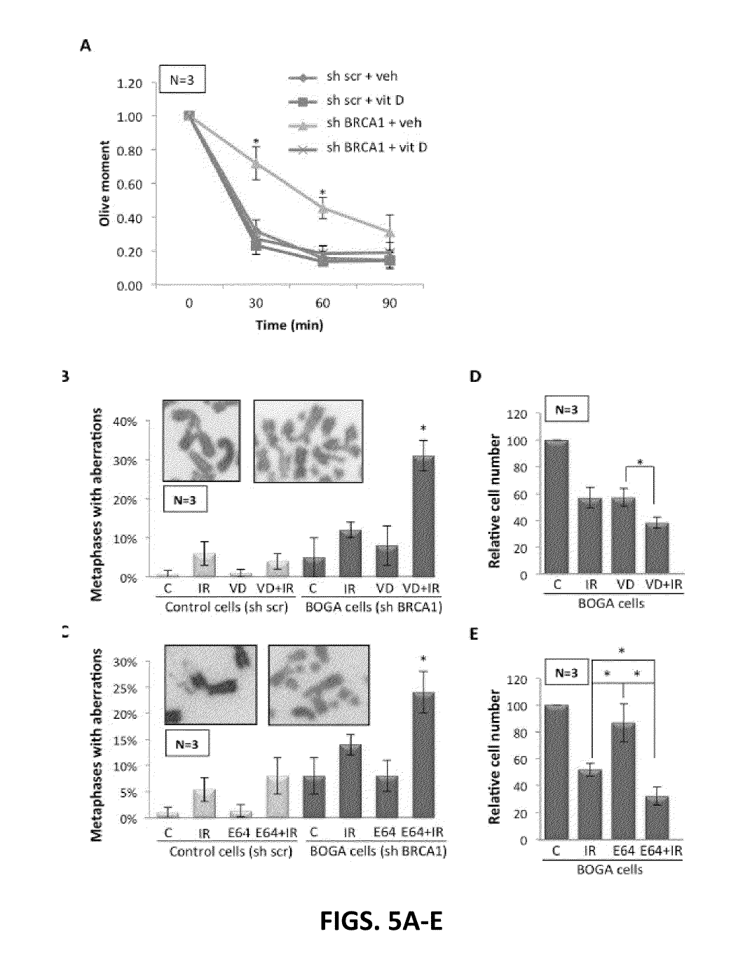

To determine the functional consequences of CTSL-mediated degradation of 53BP1 in BRCA1-deficient cells, the inventor evaluated the kinetics of DNA DSBs repair by performing comet assays under neutral, non-denaturing conditions (Olive et al., 1990). FIG. 5A shows that BOGA cells exhibit defects in the fast phase of repair corresponding to classical NHEJ (Iliakis et al., 2004), consistent with the inventor's previous finding that upregulation of CTSL in MEFs leads to defects in the fast phase of repair through degradation of 53BP1 (Gonzalez-Suarez et al., 2011). Furthermore, inhibition of CTSL with vitamin D rescues the kinetics of DNA DSBs repair, mirroring control cells (FIG. 5A). These results suggest that CTSL-mediated degradation of 53BP1 hinders NHEJ in BRCA1-deficient cells. However, these cells are still able to repair DSBs although at a lower rate, suggesting that repair by HR or alternative-NHEJ might remain relatively intact. This is consistent with RAD51 foci being able to form early post-IR.

Cells deficient in HR become dependent on alternative pathways of DNA DSBs repair, which often form complex chromosomal aberrations that trigger cell cycle arrest or death. Loss of 53BP1 is sufficient to reduce the extent of aberrant chromosome structures of BRCA1-deficient cells (Bunting et al., 2010). Here, the inventor determined whether stabilization of 53BP1 in BRCA1-deficient cells exacerbates the extent of genomic instability after IR by analyzing chromosomal aberrations in metaphase spreads. She did not find a profound increase in chromosome aberrations after IR in BOGA cells (FIG. 5B), in agreement with the deficiency in BRCA1 and 53BP1. However, stabilization of 53BP1 in this context by vitamin D treatment significantly increased the percentage of metaphases with aberrant chromosomes after IR. Similarly, stabilization of 53BP1 in BOGA cells by treatment with the cathepsin inhibitor E-64 markedly increased genomic instability after IR (FIG. 5C). Consistent with the increase in genomic instability, treatment of BOGA cells with vitamin D or E-64 significantly reduced their recovery from IR (FIGS. 5D-E). Thus, CTSL inhibition could represent a novel strategy to induce radiosensitivity in specific types of breast tumors.

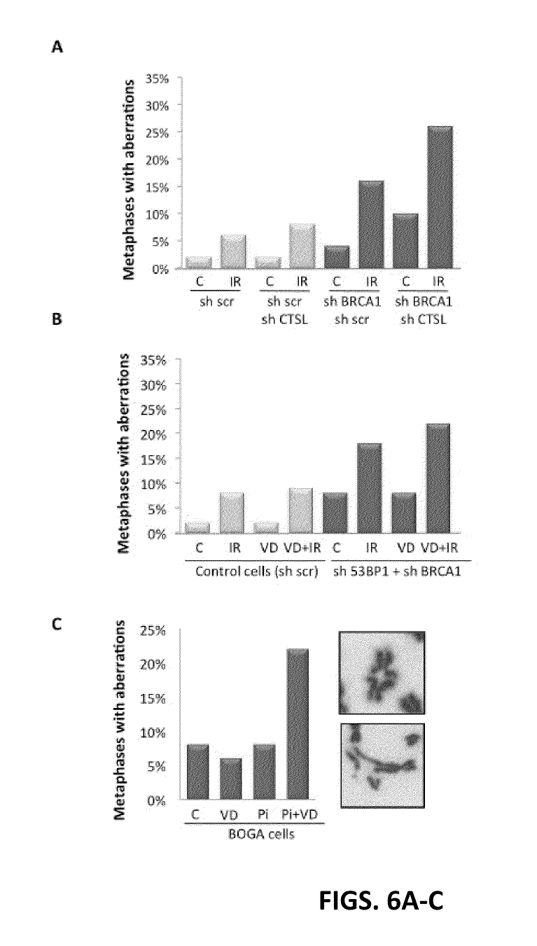

To confirm that inhibition of CTSL-mediated degradation of 53BP1 is responsible for the increase in chromosomal aberrations after IR, the inventor analyzed genomic instability in BOGA cells depleted of CTSL. These cells show a marked increases in chromosomal aberrations after IR, when compared to cells deficient in either BRCA1 or CTSL alone (FIG. 6A). Furthermore, the inventor determined if the effect of vitamin D increasing genomic instability after IR in BOGA cells is mediated by 53BP1 by monitoring chromosomal aberrations in cells double-depleted of 53BP1 and BRCA1. In these cells the combination of vitamin D and IR does not result in such profound increased in genomic instability (FIG. 6B) as observed in BOGA cells (FIG. 5B). These results demonstrate that vitamin D exerts its effect in part by stabilizing 53BP1 levels and that the extent of CTSL-mediated degradation of 53BP1 is a key determinant of the ability of BRCA1-deficient cells to deal with the DNA damage generated by IR and putatively other genotoxic agents.

BRCA1-deficient cells are exquisitely sensitive to PARPi (Bryant et al., 2005; Drew et al., 2010). Importantly, loss of 53BP1 reduces the sensitivity of these cells to PARPi (Aly and Ganesan, 2011; Bunting et al., 2010). The inventor assessed whether stabilization of 53BP1 in BOGA cells would increase the extent of genomic instability induced by PARPi. As shown in FIG. 6C, treatment of BOGA cells with PARPi does not result in profound genomic instability, in agreement with the resistance of cells double-deficient in 53BP1 and BRCA1 to this treatment. Interestingly, stabilization of 53BP1 by vitamin D increases the extent of chromosomal aberrations in response to PARPi. These data suggest that inhibition of CTSL-mediated degradation of 53BP1 could induce sensitivity to PARPi.

Increased Levels of Nuclear CTSL in TNBC and Tumors from Patients with BRCA1-Germline Mutations.

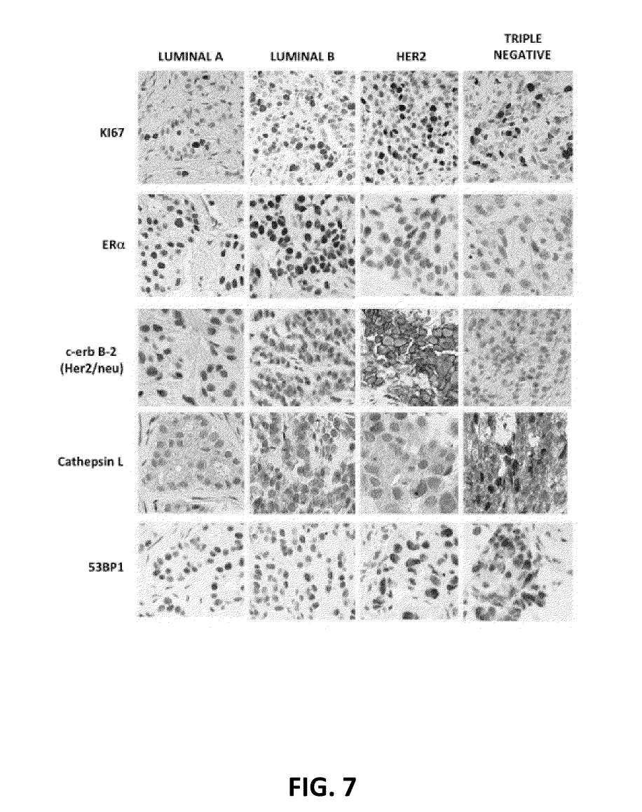

Although CTSL is one of the most abundant proteases in the endosomal/lysosomal compartment, it has also been identified in the nucleus (Duncan et al., 2008; Goulet et al., 2004). The inventor previously demonstrated that upregulation of CTSL leads to accumulation of the protease in the nucleus and degradation of 53BP1 (Gonzalez-Suarez et al., 2011). Recent studies demonstrated that loss of 53BP1 is more frequent in Triple-Negative and BRCA1 mutated human breast cancers (Bouwman et al., 2010). Here, the inventor determined whether upregulation of CTSL occurs in human breast cancers and if it correlates with decreased levels of 53BP1. In addition, given the inhibitory effect of vitamin D on this pathway, the inventor monitored the levels of vitamin D receptor (VDR), which mediates most of vitamin D's cellular effects. The inventor performed immunohistochemical (IHC) analyses of multitumor tissue microarrays (TMA) constructed with tissue from 249 patients with sporadic breast cancer (FIG. 7 and Table 1) classified into four molecular subtypes: Luminal A, Luminal B, Her2, and Triple-Negative. Immunohistochemical scores (Hscores ranging from 0 to 300) for Ki67, ER, CTSL, 53BP1, and VDR provided a semi-quantitative measurement of their expression for each tumor subtype (Pallares et al., 2009). As shown in FIG. 7, staining of CTSL was both cytoplasmic and nuclear while 53BP1 staining was only nuclear. Table 1 summarizes the immunohistochemical results. Whereas cytoplasmic CTSL Hscores were similar in all tumor subtypes, nuclear CTSL Hscores were markedly enhanced in TN tumors. In agreement with the in vitro findings, these high nuclear CTSL Hscores concur with lower 53BP1 Hscores in TNBC compared to all other tumor types. Furthermore, the inventor used the median nuclear Hscores for CTSL and 53BP1 of 0 and 150 respectively as cut-off points with identical statistical power to confirm these significant differences in CTSL and 53BP1 expression among molecular tumor types. Again, statistically significant differences were obtained, and TNBC emerged from this analysis as a remarkably different tumor subtype. Table 2 shows that 60% of TN tumors elicited Hscores for nuclear CTSL>0, a frequency more than 2-fold higher than for any other molecular type (p=0.0013). Also, 75% of TN tumors expressed 53BP1 Hscores below 150 compared to 40% of Luminal A, 49% of Luminal B, and 39% of Her2 tumors (p=0.0049). These data clearly show that higher expression of nuclear CTSL as well as lower expression of 53BP1 is significantly more associated with TNBC than any other molecular types of breast cancer. Thus, the inventor has identified nuclear CTSL as a novel biomarker for subsets of TNBC patients. Importantly, this new signature (high nuclear CTSL and low 53BP1) could serve to stratify TNBC patients.

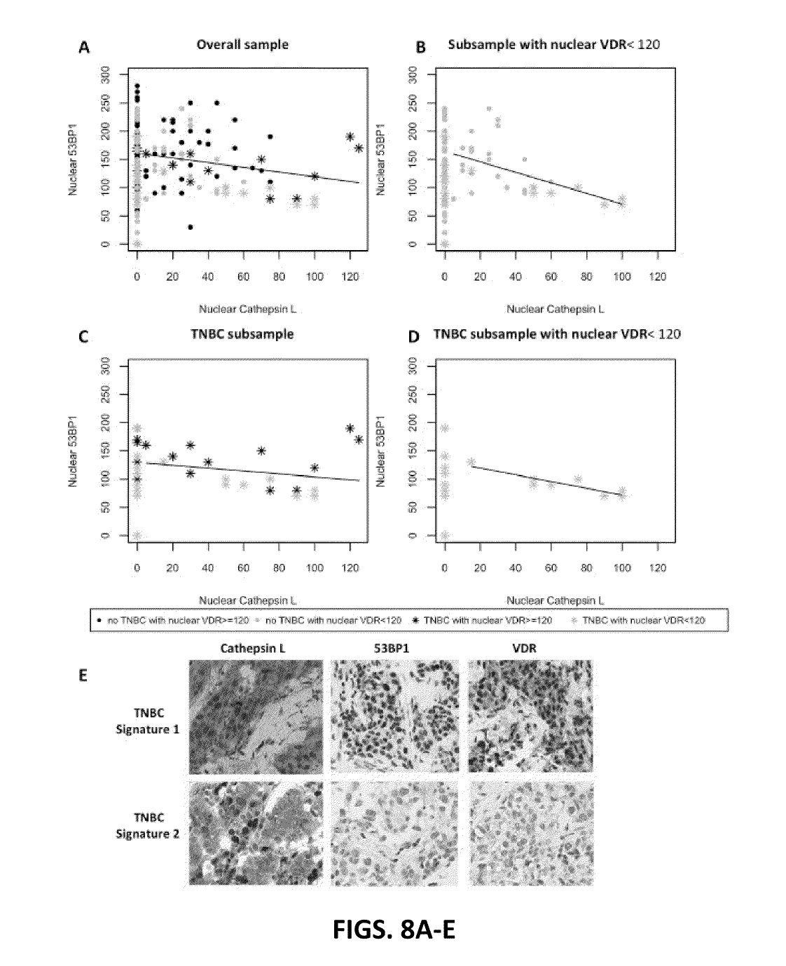

Next, the inventor analyzed breast tumors from patients with germline mutations in BRCA1 (n=18) or BRCA2 (n=14) for levels of nuclear CTSL and 53BP1 by IHC (Table 3). In comparison with the subsample of sporadic TNBC, tumors from patients with BRCA1 germline mutations elicited the same high Hscores for nuclear CTSL (p=0.95) and low Hscores for 53BP1 (p=1). In contrast, tumors from patients with BRCA2 germline mutations had nuclear CTSL Hscores similar to those in all molecular subtypes of sporadic tumors and significantly lower than BRCA1 germline tumors. Accordingly, 53BP1 Hscores were higher in tumors from patients with BRCA2 germline mutations than in BRCA1-related tumors or all molecular subtypes of sporadic tumors. These results support in vitro data for a role of CTSL in the degradation of 53BP1 in BRCA1-deficient cells. Importantly, FIG. 8A shows a statistically significant inverse linear correlation between Hscores for nuclear 53BP1 and CTSL in all tumor subtypes with a positive nuclear CTSL expression. However, a coefficient of determination of only 6.6% indicates that there is a 93.4% of the variability in 53BP1 Hscores that cannot be accounted for by increases in nuclear CTSL. These results suggest that additional factors might contribute to CTSL-mediated degradation of 53BP1 in these tumors. Identifying these factors could help to discriminate subsets of patients in which this pathway is activated.

Previous studies in human colon cancer cells showed a correlation between expression of vitamin D receptor (VDR) and cystatin D, an inhibitor of several cathepsins including CTSL (Alvarez-Diaz et al., 2009), and upregulation of cystatin D by vitamin D. In addition, in vitro data show that vitamin D inhibits CTSL-mediated degradation of 53BP1. Vitamin D actions require a functional nuclear VDR (Dusso et al., 2005). Because VDR levels are reduced in several human cancers and the loss of BRCA1 causes defective VDR translocation to the nucleus in osteosarcoma cells (Deng et al., 2009), the inventors hypothesized that there might be a threshold for nuclear VDR required to inhibit CTSL-mediated degradation of 53BP1, which in turn could explain the signature of tumors with high Hscores for both nuclear CTSL and 53BP1. The inventor's hypothesis is supported by the findings of a direct linear correlation between nuclear levels of VDR and 53BP1 for all 249 tumor types (Pearson Correlation r=0.238; p=0.0002). Analyzing the linear relationship for those tumors with nuclear VDR expression below the median HSC of 120 obtained in the 249 sporadic tumors (FIG. 8B), the inventor finds an increase in the slope of the linear regression of nuclear 53BP1 and CTSL Hscores (from −0.41 to −0.93) as well as increased coefficient of determination (from 6.6% to 29.2%). Furthermore, when the correlation between 53BP1 and CTSL was examined exclusively in TNBC, the non-significant correlation depicted in FIG. 8C becomes highly significant when only tumors with VDR<120 are examined (p<0.0027), as seen in FIG. 8D, with a coefficient of determination of 80.2%. Indeed, the outliers in FIG. 8C correspond to patients with VDR Hscores far above 120, which maintain high Hscores for nuclear 53BP1 despite Hscores for nuclear CTSL above 100 (FIG. 8E, top panels). The bottom panels of FIG. 8E show the most common biomarker signature found in TNBC and BRCA1-related tumors. Furthermore, average nuclear Hscores for the VDR are lower in tumors from patients with germline BRCA1 mutations compared to TNBC (66 vs.114, with p<0.009) (Tables 1 and 3A), suggesting that loss of BRCA1 may also impair VDR translocation to the nucleus in breast cancer. The cytosolic and nuclear VDR Hscores in BRCA2 germline mutations depicted in Table 3A suggest that only BRCA1 mutations impact VDR translocation to the nucleus resulting in less inhibition of CTSL and degradation of 53BP1.

In summary, this study reveals a new triple biomarker signature—nuclear VDR, CTSL, and 53BP1-for stratification of patients with TNBC (in which BRCA-1 is frequently somatically altered) and tumors from patients with BRCA1 germline mutations (Tables 2 and 3B). Based on these in vitro data, this signature could potentially be used as predictor of the response of these specific tumors to DNA damaging therapeutic strategies such as radiation, crosslinking reagents, and PARP inhibitors.

TABLE 1
IMMUNOHISTOCHEMICAL ANALYSIS OF CTSL, 53BP1 AND
VDR EXPRESSION IN SPORADIC HUMAN BREAST CANCER.

| | | H score | | | Kruskal-Wallis |
|---|---|---|---|---|---|
| Proteins | Molecular Type | Mean (SD) | Median [P25, P75] | Min-Max | test p-value |
| Citoplasmatic | Luminal A | 137 (36.6) | 135 [110, 160] | 50-205 | 0.50 |
| Cathepsin L | Luminal B | 134 (35.2) | 130 [110, 155] | 75-230 | |
| | Her2₍₋₁₎ | 141 (43.1) | 135 [110, 171] | 50-230 | |
| | Triple Negative₍₋₁₎ | 145 (35.2) | 140 [120, 175] | 80-200 | |
| Nuclear | Luminal A | 8 (18.0) | 0 [0, 0] | 0-90 | <.0001 |
| Cathepsin L | Luminal B | 8 (14.9) | 0 [0, 15] | 0-75 | |
| | Her2₍₋₁₎ | 9 (18.3) | 0 [0, 5] | 0-75 | |
| | Triple Negative₍₋₁₎ | 42 (44.3) | 30 [0, 83] | 0-125 | |
| 53BP1 | Luminal A | 155 (56.5) | 160 [120, 200] | 0-270 | 0.0002 |
| | Luminal B | 150 (45.8) | 150 [120, 170] | 40-280 | |
| | Her2₍₋₄₎ | 154 (58.1) | 150 [110, 200] | 20-300 | |
| | Triple Negative | 112 (44.4) | 105 [80, 143] | 0-190 | |
| Citoplasmatic | Luminal A₍₋₄₎ | 50 (50.9) | 50 [0, 90] | 0-185 | 0.22 |
| VDR | Luminal B₍₋₄₎ | 52 (50.8) | 50 [0, 80] | 0-190 | |
| | Her2₍₋₂₎ | 53 (48.8) | 50 [0, 95] | 0-160 | |
| | Triple Negative₍₋₃₎ | 69 (50.6) | 50 [20, 110] | 0-170 | |
| Nuclear | Luminal A₍₋₄₎ | 121 (68.2) | 110 [100, 170] | 0-300 | 0.78 |
| VDR | Luminal B₍₋₄₎ | 125 (65.2) | 130 [90, 160] | 0-300 | |
| | Her2₍₋₂₎ | 122 (73.3) | 125 [95, 160] | 0-300 | |
| | Triple Negative₍₋₃₎ | 114 (62.3) | 110 [80, 150] | 0-270 | |

Values are Mean and Median Hscores for nuclear and/or cytosolic CTSL, 53BP1 and VDR per tumor molecular types. Dispersion is assessed by standard deviation (SD) and percentiles 25 and 75 ([P25,P75]); Min-Max denotes Minimal and Maximal Values within the tumor subtype. (−X) represents X missing values in antigen expression per molecular type due to insufficient specimen. Bolded Hscore values highlight the molecular subtype responsible for the statistical significant difference identified with the Kruskal-Wallis Test that compares all molecular types (highlighted by a bold p value in case of any significant difference among them).

TABLE 2
FREQUENCY OF CTSL, 53BP1 AND VDR EXPRESSION WITHIN
MOLECULAR SUBTYPE RELATIVE TO THE MEDIAN VALUES IN
SPORADIC HUMAN BREAST CANCER

| | | | Fisher exact |
|---|---|---|---|
| Proteins | Molecular Type | n (%) | test p-value |
| Nuclear | Luminal A | 23 (23.2) | 0.0013 |
| Cathepsin L | Luminal B | 22 (31.9) | |
| >0 | Her2₍₋₁₎ | 12 (27.3) | |
| | Triple Negative₍₋₁₎ | 21 (60.0) | |
| 53BP1 | Luminal A | 40 (40.4) | 0.0049 |
| <150 | Luminal B | 34 (49.3) | |
| | Her2₍₋₄₎ | 20 (48.8) | |
| | Triple Negative | 27 (75.0) | |
| Nuclear | Luminal A | 49 (51.6) | 0.34 |
| VDR | Luminal B | 25 (38.5) | |
| <120 | Her2₍₋₄₎ | 18 (41.9) | |
| | Triple Negative | 17 (51.5) | |

Values denote the absolute (n) and the relative (%) frequencies of tumors with Hscore values above (nuclear CTSL) or below (nuclear 53BP1 and VDR) the median Hscore values for each protein in the overall population of sporadic breast cancer. Bolded Hscore values highlight the molecular subtype responsible for the statistical significant difference identified with the Fisher Exact Test that compares all molecular types (highlighted by a bold p value in case of any significant difference among them).

TABLE 3A
IMMUNOHISTOCHEMICAL ANALYSIS OF CTSL, 53BP1 AND
VDR EXPRESSION IN TUMORS FROM PATIENTS WITH BRCA1 OR BRCA2
GERMLINE MUTATIONS

| | | H score | | | | |
|---|---|---|---|---|---|---|
| | Mutation | | | | M-W p-value | M-W p-value |
| Proteins | Type | Mean (SD) | Median [P25, P75] | Min-Max | (vs. Sporadics) | (BRCA1 vs. 2) |
| Citoplasmatic | BRCA1₍₋₄₎ | 119 (34.2) | 110 [100, 138] | 60-190 | 0.0563 | 0.88 |
| Cathepsin L | BRCA2₍₋₁₎ | 118 (17.2) | 120 [100, 120] | 100-150 | 0.0437 | |
| Nuclear | BRCA1₍₋₄₎ | 38 (45.2) | 30 [15, 45] | 0-180 | 0.0001 | 0.0494 |
| Cathepsin L | BRCA2₍₋₁₎ | 15 (22.7) | 4 [0, 15] | 0-75 | 0.19 | |
| 53BP1 | BRCA1₍₋₁₎ | 111 (28.4) | 115 [100, 125] | 70-175 | 0.0016 | 0.0001 |
| | BRCA2₍₋₁₎ | 198 (43.6) | 210 [185, 220] | 110-270 | 0.0008 | |
| Citoplasmatic | BRCA1 | 86 (38.5) | 100 [50, 100] | 0-150 | 0.0048 | 0.0011 |
| VDR | BRCA2₍₋₃₎ | 145 (36.7) | 150 [115, 165] | 100-200 | <.0001 | |
| Nuclear | BRCA1 | 66 (52.9) | 53 [27, 100] | 0-180 | 0.0010 | 0.0001 |
| VDR | BRCA2₍₋₃₎ | 175 (57.8) | 170 [135, 193] | 110-300 | 0.0074 | |

TABLE 3B
IMMUNOHISTOCHEMICAL ANALYSIS OF CTSL, 53BP1 AND
VDR EXPRESSION IN TUMORS FROM PATIENTS WITH
BRCA1 OR BRCA2 GERMLINE MUTATIONS

| | | | Fisher exact | Fisher exact |
|---|---|---|---|---|
| | | | test | test |
| | Mutation | | p-value | p-value |
| Proteins | Type | n (%) | (vs. Sporadics) | (BRCA1 vs. 2) |
| Nuclear | BRCA1₍₋₄₎ | 12 (85.7) | 0.0001 | 0.10 |
| Cathepsin | BRCA2₍₋₁₎ | 7 (53.8) | 0.13 | |
| L >0 | | | | |
| 53BP1 <150 | BRCA1₍₋₁₎ | 15 (88.2) | 0.0019 | 0.0001 |
| | BRCA2₍₋₁₎ | 2 (15.4) | 0.0210 | |
| Nuclear | BRCA1₍₋₁₎ | 15 (83.3) | 0.0027 | 0.0051 |
| VDR <120 | BRCA2₍₋₁₎ | 3 (27.3) | 0.35 | |

(TABLE 3A) Values are Mean and Median Hscores for nuclear and/or cytosolic CTSL, 53BP1 and VDR in tumors with BRCA1 or BRCA2 germline mutations. Dispersion is assessed by standard deviation (SD) and percentiles 25 and 75 ([P25,P75]); Min-Max denotes minimal and Maximal Values within the tumor subtype. (−X) represents X missing values in antigen expression per molecular type due to insufficient specimen. Bolded Hscore values highlight the statistical significance of differences measured by Mann-Whitney test (M−) between each tumor mutation subtype vs. the overall population of sporadic breast cancer (sporadics) or between tumors with BRCA1 vs. BRCA2 germline mutations (BRCA1 vs. (2). Significant differences are highlighted with bold p value.
(TABLE 3B) Values denote the absolute (n) and the relative (%) frequencies of tumors with Hscore values above (nuclear CTSL) or below (nuclear 53BP1 and VDR) the median Hscore values for each protein in tumors with BRCA1 or BRCA2 germline mutations. Bolded Hscore values highlight the statistical significant difference measured by the bold p value from Fisher exact test comparing a molecular subtype with either the overall population of sporadic breast cancer (sporadics) or between tumors with BRCA1 vs. BRCA2 germline mutations (BRCA1 vs. 2).

### Example 3

### Discussion

Breast cancers classified as Triple-Negative (TN) or BRCA1-deficient are among the most aggressive and difficult to treat. These tumors harbor similar DNA repair deficiencies and gene expression profiles (Foulkes et al., 2010). Of particular relevance is the loss of BRCA1 function and decrease in 53BP1 levels, two factors with a decisive role in the choice of DNA DSBs repair mechanisms: HR or NHEJ (Bouwman et al., 2010). Recent landmark studies demonstrated that loss of 53BP1 allows survival of BRCA1-deficient cells and induces their resistance to DNA damaging therapeutic strategies (Bothmer et al., 2010; Bouwman et al., 2010; Bunting et al., 2010; Cao et al., 2009). Thus, stabilization of 53BP1 levels represents a new promising strategy for the treatment of these cancers. However, prior to this study, no information was available about how the levels of 53BP1 mRNA and/or protein are downregulated in breast tumor cells.

This study demonstrates that upregulation of CTSL is a mechanism responsible for lowering 53BP1 protein levels in BRCA1-deficient cells, allowing bypass of the characteristic growth arrest upon loss of BRCA1 function (FIG. 7). It is possible that other mechanisms such as degradation of 53BP1 by the proteasome also contribute to 53BP1 reduction in BRCA1-deficient tumor cells. In addition, previous studies showed that subsets of BRCA1-mutated tumors also exhibit reduced 53BP1 mRNA levels (Bouwman et al., 2010), indicating that different mechanisms can be activated in BRCA1-deficient cells to lower 53BP1 levels and ensure survival. Importantly, depletion of CTSL or inhibition of its activity stabilizes 53BP1 protein levels and induces genomic instability in BRCA1-deficient cells following IR or treatment with PARPi.

Furthermore, the inventor shows a highly significant negative correlation between 53BP1 and nuclear CTSL>0 Hscores. The highest median levels of nuclear CTSL concur with the lowest levels of 53BP1 in a subset of TNBC and tumors from patients with BRCA1 germline mutations and with low nuclear VDR levels. This study has revealed a new pathway, activated upon loss of BRCA1 function, which is anticipated to contribute to the progression of breast cancers with the poorest prognosis. Inhibition of this pathway by treatment with vitamin D or cathepsin inhibitors could provide a new therapeutic strategy for breast cancer. Importantly, the status of the pathway offers great potential as a predictive biomarker for response to therapy.

CTSL: a New Target of BRCA1 with a Function in DNA Repair.

CTSL is one of the most abundant proteases in the endosomal/lysosomal compartment and has also been identified in the nucleus (Duncan et al., 2008; Goulet et al., 2004). Upregulation of CTSL is a hallmark of a variety of cancers and has been correlated with increased invasiveness, metastasis, and overall degree of malignancy (Gocheva and Joyce, 2007; Jedeszko and Sloane, 2004; Skrzydlewska et al., 2005). Thus, inhibition of CTSL activity is considered a promising strategy for cancer treatment (Lankelma et al., 2010). In addition to the previously reported effects of CTSL upregulation on the degradation of extracellular matrix components and cell-adhesion molecules, studies in the inventor's laboratory showed that CTSL upregulation leads to accumulation of the protease in the nucleus and degradation of nuclear factors with key roles in cell cycle regulation—Rb family members and DNA repair—53BP1−. As a consequence, upregulation of CTSL leads to defects in DNA DSBs repair (Gonzalez-Suarez et al., 2011). The present study identifies CTSL as a new target of BRCA1 with an important function in regulating mechanisms of DNA DSBs repair. Several lines of evidence implicate BRCA1 in transcriptional regulation and chromatin remodeling (Bochar et al., 2000; Mullan et al., 2006), as well as in the maintenance of heterochromatin silencing (Zhu et al., 2011).

Interestingly, epigenetic mechanisms such as DNA methylation and expression of miRNAs have been linked to BRCA1 function (Kawai and Amano, 2012; Shukla et al., 2010; Tanic et al., 2011). Although the mechanism by which BRCA1-deficient cells activate CTSL is unknown, the latency in the activation of this pathway indicates that CTSL is not a direct transcriptional target of BRCA1. Rather, the inventor speculates that the loss of BRCA1 might result in alterations in chromatin structure that either make the CTSL gene more permissive to transcriptional activation over time, and/or alter the stability of CTSL mRNAs possibly via regulatory miRNAs. Those BRCA1-deficient cells that are able to activate CTSL-mediated degradation of 53BP1 would be poised to continue proliferation. This notion is supported by the inventor's data showing that prior depletion of 53BP1 prevents the characteristic growth arrest that follows BRCA1 depletion.

Vitamin D and Cathepsin Inhibitors can Modulate DNA DSBs Repair Choice.

The inventor's previous studies in mouse cells and the present study in human breast cancer cells reveal an unprecedented role for vitamin D and cathepsin inhibitors in the regulation of DNA DSBs repair choice. By stabilizing 53BP1 protein levels in the context of BRCA1 deficiency, CTSL inhibitors facilitate repair of DSBs by NHEJ while inhibiting HR. Importantly, these data indicate that both vitamin D and cathepsin inhibitors impact on 53BP1 stability, especially in cells that upregulate CTSL, i.e., BRCA1-deficient cells, showing a lesser effect in cells with normal CTSL expression. This is likely due to the fact that upregulation of CTSL leads to an increase in the levels of nuclear CTSL, which is low relative to other cellular compartments in normal cells (Gonzalez-Suarez et al., 2011). This notion is supported by the analysis of TMA, which showed that while all breast tumor types exhibit cytoplasmic CTSL, a subset of TNBC and BRCA1-deficient tumors present with high levels of nuclear CTSL.

Interestingly, a percentage of these tumors are also deficient in nuclear VDR and 53BP1. The ability to impact the choice of DNA DSBs repair pathway could have profound consequences for cancer therapy. In tumor cells that activate CTSL-mediated degradation of 53BP1 as a means to ensure proliferation and survival, cathepsin inhibition could stabilize 53BP1, increase genomic instability, and induce growth arrest and/or cell death, especially in combination with IR or PARPi. Thus, treatment with vitamin D or CTSL inhibitors could represent a new therapeutic strategy for specific types of breast tumors, if they can be identified.

Nuclear Levels of CTSL, 53BP1 and VDR: a New Predictive Biomarker Signature for Drug Response?

The analysis of TMA suggests that levels of nuclear CTSL represent a new positive biomarker for subsets of tumors having abnormalities in BRCA-1, including TNBC (currently defined solely by the absence of Her2/neu, estrogen and progesterone receptors, and frequently showing somatic alterations of BRCA-1 function), and tumors arising in association with germline mutations in BRCA-1. Similarly, the inventor finds that nuclear VDR levels correlate linearly with 53BP1 content in all tumor subtypes, and the lowest nuclear VDR levels correspond to TNBC and tumors from patients with BRCA1 germline mutations. The finding of the strong negative correlation between reductions in nuclear 53BP1 with increases in nuclear CTSL levels with coefficient of determination of 80% in TNBC with nuclear VDR<120 is very interesting, as TNBC is associated with severe vitamin D deficiency (Peppone et al., 2011). Furthermore, previous studies had shown a role for VDR in the upregulation of cathepsin inhibitors (Alvarez-Diaz et al., 2009). Thus, it is tempting to speculate that vitamin D interventions could lead to VDR-induced expression of cystatins and the attenuation of CTSL-mediated degradation of 53BP1. Future studies testing this hypothesis might lead to new strategies of targeted therapy.

The combination of low nuclear VDR or high nuclear CTSL with low 53BP1 levels offers great potential for the stratification of BRCA1-deficient and TNBC patients into different subgroups and as a predictive biomarker for the response of these patients to current therapies. In particular, the use of PARPi as single agents or in combination with radiation or chemotherapy is a leading strategy for breast cancer management, especially for HR-deficient tumors (Drew et al., 2010; Farmer et al., 2005; Fong et al., 2009; Helleday et al., 2005; Tutt et al., 2010). However, a significant fraction of these cancers acquire resistance to PARPi. Recent studies in cell culture and mouse models demonstrated that loss of 53BP1 reduces the sensitivity of BRCA1-deficient cells to PARPi (Bunting et al., 2010).

This study suggests that BRCA1-deficient and TNBC patients that exhibit low nuclear VDR or high nuclear CTSL and low 53BP1 levels are likely to be proficient in HR and resistant to PARPi. Therefore, these patients will not benefit from this specific treatment unless levels of 53BP1 are stabilized. For these types of tumors, treatment with vitamin D or CTSL inhibitors to stabilize 53BP1 levels in combination with PARPi might result in the most effective therapy.

All of the methods and apparatus disclosed and claimed herein can be made and executed without undue experimentation in light of the present disclosure. While the compositions and methods of this invention have been described in terms of particular embodiments, it will be apparent to those of skill in the art that variations may be applied to the methods and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,277,437
U.S. Pat. No. 4,275,149
U.S. Pat. No. 4,366,241
U.S. Pat. No. 4,683,195
U.S. Pat. No. 4,683,202
U.S. Pat. No. 4,800,159
U.S. Pat. No. 4,879,236
U.S. Pat. No. 4,883,750
U.S. Pat. No. 4,981,957
U.S. Pat. No. 5,118,800
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,319,080
U.S. Pat. No. 5,324,633
U.S. Pat. No. 5,359,044
U.S. Pat. No. 5,384,261
U.S. Pat. No. 5,393,878
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,514,785
U.S. Pat. No. 5,519,134
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,567,811
U.S. Pat. No. 5,571,639
U.S. Pat. No. 5,576,427
U.S. Pat. No. 5,580,726
U.S. Pat. No. 5,580,732
U.S. Pat. No. 5,591,722
U.S. Pat. No. 5,593,839
U.S. Pat. No. 5,597,909
U.S. Pat. No. 5,599,672
U.S. Pat. No. 5,599,695
U.S. Pat. No. 5,610,300
U.S. Pat. No. 5,610,287
U.S. Pat. No. 5,624,711
U.S. Pat. No. 5,627,053
U.S. Pat. No. 5,631,134
U.S. Pat. No. 5,639,603
U.S. Pat. No. 5,639,873
U.S. Pat. No. 5,646,265
U.S. Pat. No. 5,654,413
U.S. Pat. No. 5,658,734
U.S. Pat. No. 5,658,873
U.S. Pat. No. 5,661,028
U.S. Pat. No. 5,665,547
U.S. Pat. No. 5,667,972
U.S. Pat. No. 5,670,633
U.S. Pat. No. 5,695,940
U.S. Pat. No. 5,700,637
U.S. Pat. No. 5,700,920
U.S. Pat. No. 5,739,169
U.S. Pat. No. 5,744,305
U.S. Pat. No. 5,757,994
U.S. Pat. No. 5,788,166
U.S. Pat. No. 5,792,747
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,838,002
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5,900,481
U.S. Pat. No. 5,902,880
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,986,258
U.S. Pat. No. 6,004,755
U.S. Pat. No. 6,087,102
U.S. Pat. No. 6,146,886
U.S. Pat. No. 6,268,490
U.S. Pat. No. 6,368,799
U.S. Pat. No. 6,383,749
U.S. Pat. No. 6,395,713
U.S. Pat. No. 6,525,191
U.S. Pat. No. 6,531,584
U.S. Pat. No. 6,600,032
U.S. Pat. No. 6,617,112
U.S. Pat. No. 6,638,717
U.S. Pat. No. 6,670,461
U.S. Pat. No. 6,720,138
U.S. Pat. No. 6,770,748
U.S. Pat. No. 6,794,499
U.S. Pat. No. 7,034,133
U.S. Pat. No. 7,053,207
U.S. Pat. RE 35,413
U.S. Patent Publn. 2003/0082807
U.S. Patent Publn. 2003/0207841
U.S. Patent Publn. 2004/0014959
U.S. Patent Publn. 2004/0143114
U.S. Patent Publn. 2004/0171570
U.S. Patent Publn. 2004/0219565
U.S. Patent Publn. 2007/0213292
U.S. Patent Publn. 2007/0287831
U.S. Patent Publn. 2008/0009439
U.S. Patent Publn. 2008/0015162
U.S. Patent Publn. 2008/0032945
Adelaide et al., Cancer Res., 67:11565-11575, 2007.
Akhtar et al., Trends in Cell Bio., 2:139, 1992.
Akhtar, In: Delivery Strategies for Antisense Oligonucleotide Therapeutics, 1995.
Akinc et al. Nat. Biotechnol., 2008 May; 26(5):561-9, 2008.
Aldrian-Herrada et al., Nucleic Acids Res., 26:4910-16, 1998.
Allerson et al., J. Med. Chem., 48:901-904, 2005.
Alli et al., Oncogene, 24:39-46, 2005.
Alo et al., Cancer, 77:474-482, 1996.
Alo et al., Tumori, 85:35-40, 1999.
Alvarez-Diaz et al., J Clin Invest. 119:2343-2358, 2009.
Aly and Ganesan, J Mol Cell Biol. 3:66-74 2011.
Ambros, Cell, 113(6):673-676, 2003.
Arap et al., Cancer Res., 55(6):1351-1354, 1995.
Austin-Ward and Villaseca, Revista Medica de Chile, 126(7):838-845, 1998.
Ausubel et al., In: Current Protocols in Molecular Biology, John, Wiley & Sons, Inc, NY, 1994; 1996.
Bahr et al., J. Mass Spectrom., 32:1111-1116, 1997.
Baichwal and Sugden, In: Gene Transfer, Kucherlapati (Ed.), Plenum Press, NY, 117-148, 1986.
Bakhshi et al., Cell, 41(3):899-906, 1985.
Bellus, J. Macromol. Sci. Pure Appl. Chem., A31(1): 1355-1376, 1994.
Bentin and Nielsen, Biochemistry, 35:8863-8869, 1996.
Bentwich et al., Nat. Genet., 37:766-770, 2005.
Bentzley et al., Anal Chem., 68(13):2141-2146, 1996.
Bhattacharyya et al., J Biol Chem. 275:23899-23903, 2000.
Bhat-Nakshatri et al., Nucleic Acids Res., 37(14):4850-4861, 2009.
Blenkiron et al., Genome Biol., 8:R214, 2007.
Boado et al. J. Pharm. Sci., 87(11):1308-15, 1998.
Boado, Adv. Drug Delivery Rev., 15:73, 1995.
Bochar et al., Cell. 102:257-265, 2000.
Bothmer et al., J Exp Med. 207:855-865, 2010.
Bouwman et al., Nat Struct Mol Biol. 17:688-695, 2010.
Braasch et al., Methods, 23(2):97-107, 2001.
Brennecke et al., Cell, 113(1):25-36, 2003.
Bryant et al., Nature. 434:913-917, 2005.
Bucknall et al., J. Am. Soc. Mass Spectrom., 13(9):1015-1027, 2002.
Bukowski et al., Clinical Cancer Res., 4(10):2337-2347, 1998.
Bunting et al., Cell. 141:243-254, 2010.
Burk et al., EMBO Rep., 9:582-589, 2008.
Caldas et al., Cancer Res., 54:3568-3573, 1994.
Caldas et al., Nat. Genet., 8(1):27-32, 1994.
Calin et al., Proc. Natl. Acad. Sci. USA, 101:11755-11760, 2004.
Calin et al., Proc. Natl. Acad. Sci. USA, 99(24):15524-15529, 2002.
Cao et al., Embo J. 25:2167-2177, 2006.
Cao et al., Mol Cell. 35:534-541, 2009.
Caprioli et al., Anal. Chem., 69:4751, 1997.
Carbonelli et al., FEMS Microbiol. Lett., 177(1):75-82, 1999.
Carrington et al. Science, 301(5631):336-338, 2003.
Chang et al., J. Exp. Med., 200:1359-1370, 2004.
Chang et al., Nature, 430(7001):785-789, 2004.
Chang et al., Proc. Natl. Acad. Sci. USA, 105:4477-4482, 2008.
Chaurand et al., Anal Chem., 71)23:5263-5270, 1990.
Chen et al., Nat Biotechnol., 19:537-542, 2001.
Chen & Greene, Mol. Cell. Biol. 5:392-401, 2004.
Chen et al., Biochem. Biophys. Res. Commun., 385:596-600, 2009.
Chen et al., Fed. of Eur. Biochem Soc., 309:115-118, 1992.
Chen et al., Genes Dev., 19:1288-1293, 2005.
Cheng et al., Cancer Res., 54(21):5547-5551, 1994.
Cho, Mol. Cancer, 6:60, 2007.
Choung et al., Biochem. Biophys. Res. Commun., 342:919-927, 2006.
Chowrira et al., J. Biol. Chem., 269(41):25856-25864, 1994.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Cocea, Biotechniques, 23(5):814-816, 1997.
Cochrane et al., Mol. Cancer Ther., 2009 [Epub ahead of print]
Coupar et al., Gene, 68:1-10, 1988.
Couture et al., Trends in Genetics, 12:510, 1996.
Cowland et al., Apmis, 115:1090-106, 2007.
Davidson et al., J. Immunother., 21(5):389-398, 1998.
De Jager et al., Semin Nucl Med 23(2):165-179, 1993.
de Ronde et al., Breast Cancer Res. Treat., 2009 [Epub ahead of print]
De Schrijver et al., Cancer Res., 63:3799-3804, 2003.
Deng et al., Mol Endocrinol. 23:226-236, 2009.
Demidov et al., ChemBiochem., 2:133-139, 2001.
Desiderio et al., J. Mass Spectrom., 35(6):725-733, 2000.
Dogini et al., J. Mol. Neurosci., 35(3):331-337, 2008.
Doolittle et al., Methods Mol Biol, 109:215-237, 1999.
Dostie et al., RNA, 9:180-186, 2003.
Drew et al., J Natl Cancer Inst. 103:334-346, 2010.
Duncan et al., Rapid Commun. Mass Spectrom., 7(12):1090-1094, 1993.
Duncan et al., Cell. 135:284-294, 2008.
Dusso, et al., Am J Physiol Renal Physiol. 289:F8-28, 2005.
Duursma et al., RNA, 14 (5):872-877, 2008.
Egholm et al., Nature, 365(6446):566-568, 1993.
Elayadi et al., Nucleic Acids Res., 29(8):1683-9, 2001.
Elmén et al., Nature, 452(7189):896-9, 2008.
Engels and Hutvagner, Oncogene, 25:6163-6169, 2006.
Esau et al. Cell Metab., 3(2):87-98, 2006.
European Appln. 320 308
European Appln. 329 822
European Appln. 373 203
European Appln. 785 280
European Appln. 799 897 Evers and Jonkers, Oncogene. 25:5885-5897, 2006.
Fabbri et al, Proc. Natl. Acad. Sci. USA, 104:15805-15810, 2007.
Farmer et al., Nature. 434:917-921, 2005.
Faruqi et al., Proc. Natl. Acad. Sci. USA, 95:1398-1403, 1998.
Fatkin et al., J. Clin. Invest., 106(11):1351-1359, 2000.
Faulstich et al., Anal. Chem., 69(21):4349-4353, 1997.
Fenn et al., Science, 246(4926):64-71, 1989.
Fernandez-Capetillo et al., Nat Cell Biol. 4:993-997, 2002.
Fodor et al., Science, 251:767-773, 1991.
Fong, Engl J Med. 361:123-134, 2009.
Foulkes et al., Engl J Med. 363:1938-1948, 2010.
Frieden et al., Nucle. Nucleo. Nucleic Acids, 22(5-8):1041-3, 2003.
Friedmann, Science, 244:1275-1281, 1989.
Frohman, In: PCR Protocols: A Guide To Methods And Applications, Academic Press, N.Y., 1990.
Gartner et al., Cancer J. 16:83-90, 2010.
GB Appln. 2 202 328
Gobom et al., Anal. Chem., 72(14):3320-3326, 2000.
Gonzalez-Suarez et al., Cell Cycle. 8:3860-3865, 2009.
Gonzalez-Suarez et al., Embo J. 30:3383-3396, 2011.
Goulet et al., Mol Cell. 14:207-219, 2004.
Gregory et al., Nat. Cell Biol., 10(5):593-601, 2008.
Griffiths-Jones et al., Nucleic Acids Res., 34:D140-144, 2006.
Gulbis and Galand, Hum Pathol 24(12):1271-1285, 1993.
Hacia et al., Nature Genet., 14:441-449, 1996.
Haeberli et al., Nucleic Acids Res., 33:3965-3975, 2005.
Hall et al., Nucleic Acids Res., 32:5991-6000, 2004.
Hall et al., Nucleic Acids Res., 34:2773-2781, 2006.
Halterman, Pharmacotherapy, 28:1255-1266, 2008.
Hanibuchi et al., Int. J. Cancer, 78(4):480-485, 1998.
Hassani et al., J. Gene Med., 7(2):198-207, 2004.
Helleday et al., Cell Cycle. 4:1176-1178, 2005.
Hellstrand et al., Acta Oncologica, 37(4):347-353, 1998.
Hermonat and Muzycska, Proc. Natl. Acad. Sci. USA, 81:6466-6470, 1984.
Hofland and Huang, Handb. Exp. Pharmacol., 137:165, 1999.
Holstege et al., EMBO J., 16:7468-7480, 1997.
Horak et al., Rapid Commun. Mass Spectrom., 15(4):241-248, 2001.
Horwich et al. J. Virol., 64:642-650, 1990.
Hoshika et al., Nucleic Acids Res., 32:3815-3825, 2004.
Hui and Hashimoto, Infection Immun., 66(11):5329-5336, 1998.
Hurteau et al., Cancer Res., 67:7972-7976, 2007.
Hurteau et al., Cell Cycle, 8(13):2064-2069, 2009.
Hussussian et al., Nat. Genet., 8(1):15-21, 1994.
Hutvagner et al., PLoS Biol., 2(4):E98, 2004.
Iliakis et al., Cytogenet Genome Res. 104:14-20, 2004.
Innis et al., Proc. Natl. Acad. Sci. USA, 85(24):9436-9440, 1988.
Inouye and Inouye, Nucleic Acids Res., 13:3101-3109, 1985.
Iorio et al., Cancer Res., 65:7065-7070, 2005.
Iorio et al., Eur. J. Cancer, 44:2753-2759, 2008.
Ishiwata et al., Chem. Phare. Bull., 43:1005, 1995.
Izant and Weintraub, Science, 229:345, 1985.
Jedeszko and Sloane, Biol Chem. 385:1017-1027, 2004.
Jensen et al., Mod. Pathol., 21:1413-1420, 2008.
Jespersen et al., Anal Chem., 71(3):660-666, 1999.
Jiang et al., J. Agric. Food Chem., 48:3305, 2000.
Jolliet-Riant and Tillement, Fundam. Clin. Pharmacol., 13:16, 1999.
Ju et al., Gene Ther., 7(19):1672-1679, 2000.
Kahl et al., J. Mol. Biol., 299:75-89, 2000.
Kaihatsu et al., Biochem., 42(47):13996-4003, 2003.
Kaihatsu et al., Chem. Biol., 11:749-758, 2004.
Kaklamani and Gradishar, Curr. Treat. Options Oncol., 7:123-128, 2006.
Kamb et al., Nat. Genet., 8(1):23-26, 1994.
Kamb et al., Science, 2674:436-440, 1994.
Kanazawa et al., Biol. Pharm. Bull., 22(4):339-346, 1999.
Kashani-Sabet et al., Antisense Res. Dev., 2:3, 1992.
Kaur et al., Biochemistry, 45(23):7347-7355, 2006.
Kawai and Amano., Journal of Cell Biology. DOI: 10.1083/jcb.201110008, 2012.
Kazmaier et al., Anesthesiology, 89(4):831-817, 1998.
Kerr et al., Br. J. Cancer, 26(4):239-257, 1972.
Kim et al., Proc. Natl. Acad. Sci. USA, 101:360-365, 2004.
Kiriazis and Kranias, Annu. Rev. Physiol., 62:321-351, 2000.
Klinakis et al., Proc. Natl. Acad. Sci. USA, 106:2359-2364, 2009.
Korpal et al., J. Biol. Chem., 283:14910-14914, 2008.
Koshkin et al., Bioorg. Med. Chem. Lett., 8(16):2219-22, 1998.
Krenz and Robbins, J. Am. Coll. Cardiol., 44:2390-2397, 2004.
Kriitzfeldt et al., Nature, 438:685-689, 2005.
Kumar et al., Biochem. Pharmacol., 55:775-783, 1998.
Kumar et al., Nat. Genet., 39:673-677, 2007.
Kwoh et al., Proc. Natl. Acad. Sci. USA, 86:1173, 1989.
Lagos-Quintana et al., Curr. Biol., 12:735-739, 2002.
Lagos-Quintana et al., Science, 294(5543):853-858, 2001.
Landgraf et al., Cell, 129:1401-1414, 2007.
Lankelma et al., Life Sci. 86:225-233, 2010.
Larsen and Nielsen, Nucl. Acids Res., 24:458-463, 1996.
Lasic et al., Chem. Rev. 95:2601, 1995.
Lasic et al., Science, 267:1275, 1995.
Lau et al., Science, 294(5543):858-862, 2001.
Law et al., Cancer Res., 68:10238-10246, 2008.
Lee and Ambros, Science, 294(5543):862-864, 2001.
Lee et al., Arch. Biochem. Biophys., 381:43-52, 2000.
Lerma et al., Mod. Pathol., 20:1200-1207, 2007.
Levenson et al., Hum. Gene Ther., 9(8):1233-1236, 1998.
Li et al., Trends Biotechnol, 18:151, 2000.
Li and Carthew, Cell, 123:1267-1277, 2005.
Li et al., Proc. Natl. Acad. Sci. USA, 102:19231-19236, 2005.
Lim et al., Science, 299:1540, 2003.
Lin et al., Intl. J. Cancer, 123:372-379, 2008.
Liu et al., Cancer Res.., 55(14):3117-3122, 1995.
Lovelace et al., J. Chromatogr., 562(1-2):573-584, 1991.
Lowery et al., Clin. Cancer Res., 14:360-365, 2008.
Lowes et al., J. Clin. Invest., 100(9):2315-2324, 1997.
Lu et al., Nature, 435:834-838, 2005.
Lui et al., Cancer Res. 67:6031-6043(2007).
Lynn et al., J. Mol. Evol., 48(5):605-614, 1999.
Marie et al., Anal. Chem., 72(20):5106-5114, 2000.
Mashima et al., Br. J. Cancer, 100:1369-1372, 2009.
Mattie et al., Mol. Cancer, 5:24, 2006.
Maurer et al., Mol. Membr. Biol., 16:129, 1999.
McGarry and Lindquist, Proc. Natl. Acad. Sci. USA, 83:399, 1986.
McMahon et al., Life Sci., 71(3):325-3, 2002.
Meche et al., J. Morphol. Embryol., 50:217-221, 2009.
Meister et al., Mol. Cell., 15:185-197, 2004.
Menendez and Lupu, Arch. Immunol. Ther. Exp. (Warsz), 52:414-426, 2004.
Menendez et al., Int. J. Cancer, 115:19-35, 2005.
Miketova et al., Mol. Biotechnol., 8(3):249-253, 1997.
Miller et al., J. Biol. Chem., 283:29897-29903, 2008.
Mirgorodskaya et al., Rapid Commun. Mass Spectrom., 14(14):1226-1232, 2000.
Mitchell et al., Ann. NY Acad. Sci., 690:153-166, 1993.
Mitchell et al., J. Clin. Oncol., 8(5):856-869, 1990.
Miyata et al., Circ. Res., 86(4):386-390, 2000.
Mollegaard et al., Proc. Natl. Acad. Sci. USA, 91:3892-3895, 1994.
Mori et al., Cancer Res., 54(13):3396-3397, 1994.
Morita et al., Bioorg. Med. Chem., 11(10):2211-26, 2003.
Morris and Carey, Rev. Endocr. Metab. Disord., 8:185-198, 2007.
Morris et al., Science, 305:1289-1292, 2004.
Morton et al., Arch. Surg., 127:392-399, 1992.
Moynahan et al., Mol Cell. 4:511-518, 1999.
Muddiman et al., Fres. J. Anal. Chem., 354:103, 1996.
Mukohara et al., Cancer Lett., 282:14-24, 2009.
Mullan et al., Oncogene. 25:5854-5863, 2006.
Nakamura et al., In: Handbook of Experimental Immunology (4^{th} Ed.), Weir et al, (eds). 1; 27, Blackwell Scientific Publ., Oxford, 1987.
Neuhausen and Marshall, Cancer Research., 54:6069-6072, 1994.
Nelson et al., Anal. Chem., 66:1408, 1994.
Nguyen et al., J. Chromatogr. A., 705(1):21-45, 1995.
Nicolas and Rubinstein, In: Vectors: A survey of molecular cloning vectors and their uses, Rodriguez and Denhardt, eds., Stoneham: Butterworth, pp. 494-513, 1988.
Nielsen et al., Science, 254:1497-1500, 1991.
Nobori et al., Nature (London), 368:753-756, 1995.
Ohara et al., Proc. Natl. Acad. Sci. USA, 86:5673-5677, 1989.
Ohkawa et al., Nucleic Acids Symp. Ser., 27:156, 1992.
Ojwang et al., Proc. Natl. Acad. Sci. USA, 89:10802, 1992.
Okamoto et al., Proc. Natl. Acad. Sci. USA, 91(23):11045-11049, 1994.
Oku et al., Biochim. Biophys. Acta, 1238:86, 1995.
Olive et al., Radiat Res. 122:86-94, 1990.
Orlow et al., Cancer Res, 54(11):2848-2851, 1994.
Ouellet et al. J. Biomed. Biotechnol., 2006(4):69616, 2006.
Pallares et al., Analytical and quantitative cytology and histology/the International Academy of Cytology [and] American Society of Cytology. 31:217-226, 2009.
Pandey and Picard, Mol. Cell Biol., 29:3783-3790, 2009.
Pardridge et al., Proc. Natl. Acad. Sci. USA, 92:5592, 1995.
Park et al., Genes Dev., 22:894-907, 2008.
Paroo and Corey, Trends Biotechnol., 22(8):390-4, 2004.
Pasquinelli and Ruvkun, Ann. Rev. Cell Dev. Biol., 18:495-513, 2002.
Paterson et al., ScientificWorld J., 8:901-904, 2008.
PCT Appln. PCT/US87/00880
PCT Appln. PCT/US89/01025
PCT Appln. WO 00/53722
PCT Appln. WO 01/38580
PCT Appln. WO 01/68255
PCT Appln. WO 03/020898
PCT Appln. WO 03/022421
PCT Appln. WO 03/023058
PCT Appln. WO 03/029485
PCT Appln. WO 03/040410
PCT Appln. WO 03/053586
PCT Appln. WO 03/066906
PCT Appln. WO 03/067217
PCT Appln. WO 03/076928
PCT Appln. WO 03/087297
PCT Appln. WO 03/091426
PCT Appln. WO 03/093810
PCT Appln. WO 03/100012
PCT Appln. WO 03/100448A1
PCT Appln. WO 04/020085
PCT Appln. WO 04/027093
PCT Appln. WO 05/021570
PCT Appln. WO 05/121371
PCT Appln. WO 05/115481
PCT Appln. WO 09/923256
PCT Appln. WO 09/936760
PCT Appln. WO 88/10315
PCT Appln. WO 89/06700
PCT Appln. WO 90/07641
PCT Appln. WO 93/17126
PCT Appln. WO 93/23569
PCT Appln. WO 94/02595
PCT Appln. WO 94/02595
PCT Appln. WO 94/14226
PCT Appln. WO 95/11995
PCT Appln. WO 95/21265
PCT Appln. WO 95/21944
PCT Appln. WO 95/35505
PCT Appln. WO 96/10390
PCT Appln. WO 96/10391
PCT Appln. WO 96/10392
PCT Appln. WO 96/31622
PCT Appln. WO 97/10365
PCT Appln. WO 97/27317
PCT Appln. WO 97/43450
PCT Appln. WO 98/39352
PCT Appln. WO 99/14226
PCT Appln. WO 99/35505
Pease et al., Proc. Natl. Acad. Sci. USA, 91:5022-5026, 1994.
Peppone et al., Breast cancer research and treatment. 127:171-177, 2011.
Pietras et al., Oncogene, 17(17):2235-2249, 1998.
Poy et al., Nature, 432:226-230, 2004.
Prakash et al., J. Med. Chem., 48:4247-4253, 2005.
Propulic et al., J. Virol., 66:1432-1441, 1992.
Puig et al., Breast Cancer Res. Treat., 109:471-479, 2008.
Qin et al., Proc. Natl. Acad. Sci. USA, 95(24):14411-14416, 1998.
Rakha and Ellis, Pathology, 41:40-47, 2009.
Rand et al., Cell, 123:621-629, 2005.
Ravindranath and Morton, Intern. Rev. Immunol., 7: 303-329, 1991.
Redwood et al., Cell Cycle. 10, 2011a.
Redwood et al., Cell Cycle. 10:2549-2560, 201 lb.
Ridgeway, In: Vectors: A Survey of Molecular Cloning Vectors and Their Uses, Rodriguez et al. (Eds.), Stoneham: Butterworth, 467-492, 1988.
Roepstorff, E X S., 88:81-97, 2000.
Rosenberg et al., Ann. Surg. 210(4):474-548, 1989.
Rosenberg et al., N. Engl. J. Med., 319:1676, 1988.
Saetrom et al., Nucleic Acids Res., 35:2333-2342, 2007.
Sambrook et al., In: Molecular cloning: a laboratory manual, 2^{nd} Ed., Cold Spring Harbor Laboratory Press, Cold Spring Harbor, N.Y., 1989.
Sambrook and Russell, Molecular Cloning: A Laboratory Manual 3^{rd} Ed., Cold Spring Harbor Laboratory Press, 2001.
Sarver et al., Science, 247:1222-1225, 1990.
Scanlon et al., Proc. Natl. Acad. Sci. USA, 88:10591-10595, 1991.
Schlegel, Cancer research. 66:5181-5189, 2006.
Schultz et al., J Cell Biol. 151:1381-1390, 2000.
Scully et al., Cell., 90:425-435, 1997a.
Scully and Livingston, Nature. 408:429-432, 2000.
Sempere et al. Cancer Res., 67(24):11612-11620, 2007.
Serrano et al., Nature, 366:704-707, 1993.
Serrano et al., Science, 267(5195):249-252, 1995.
Sewer et al., Bioinformatics, 6:267, 2005.
Shoemaker et al., Nature Genetics, 14:450-456, 1996.
Singh et al., J. Biol. Chem., 273:20354-20362, 1998.
Shukla et al., 2010
Skrzydlewska et al., World J Gastroenterol. 11:1251-1266, 2005.
Snouwaert et al., Oncogene. 18:7900-790, 1999.
Soutschek et al., Nature, 432(7014):173-178, 2004.
Stockmans et al., Curr. Opin. Oncol., 20:614-620, 2008.
Stoeckli et al., Nat. Med., 7(4):493-496, 2001.
Summerton and Weller, Antisense Nucleic Acid Drug Dev., 7(3):187-95, 1997.
Sung et al., J Biol Chem. 278:42729-42732, 2003.
Swinnen et al., Biochem. Biophys. Res. Commun., 302:898-903, 2003.
Taira et al., Nucleic Acids Res., 19:5125, 1991.
Takach et al., J. Protein Chem., 16:363, 1997.
Tanic et al., Breast cancer research and treatment., 2011.
Taylor and Gercel-Taylor, Gynecol. Oncol., 110:13-21, 2008.
Temin, In: Gene Transfer, Kucherlapati (Ed.), NY, Plenum Press, 149-188, 1986.
Thompson et al., Nature Genet., 9:444-450, 1995.
Tsujimoto and Croce, Proc. Natl. Acad. Sci. USA, 83(14):5214-5218, 1986.
Tsujimoto et al., Nature, 315:340-343, 1985.
Turner and J. S. Reis-Filho., Oncogene., 25:5846-5853, 2006.
Tutt et al., Lancet. 376:235-244, 2010.
Tyler et al. Proc. Natl. Acad. Sci. USA, 8; 96(12):7053-8, 1999b.
Tyler et al., Am. J. Physiol., 277(6 Pt 1):L1199-204, 1999a.
UK Appln. 8 803 000
Urban-Klein, Gene Ther., 12:461-6, 2005.
Vazquez-Martin et al., J. Cell Biochem., 105:1147-1152, 2008.
Ventura et al., Nucleic Acids Res., 21:3249, 1993.
Villanueva et al., Enzyme Microb. Technol., 29:99, 1999.
Wahlestedt et al., Proc. Natl. Acad. Sci. USA, 97:5633-5638, 2000
Walker et al., Proc. Natl. Acad. Sci. USA, 89:392-396, 1992.
Wang et al., J. Agric. Food. Chem., 47:1549, 1999.
Wang et al. Science, 298:1435-1438, 2002.
Ward et al., Mol Cell Biol. 23:2556-2563, 2003.
Watanabe et al., Genes Dev., 20:1732-1743, 2006.
Webster et al., J. Biol. Chem., 284:5731-5741, 2009.
Weerasinghe et al., J. Virol., 65:5531, 1991.
Wittman et al., Biotechnol. Bioeng., 72:642, 2001.
Wooster and Weber, Engl J Med., 348:2339-2347, 2003.
Wu et al., Biochem. Biophys., Res. Commun., 233(1):221-226, 1997.
Wu et al., Anal. Chem., 70:456A, 1998.
Wu et al., Biochim. Biophys. Acta, 1466:315-327, 2000
Xie et al., Mol Cell. 28:1045-1057, 2007.
Xu et al., Mol Cell Biol. 21:3445-3450, 2001.
Xu et al., Curr. Biol., 13(9):790-795, 2003.
Yang et al., J. Agric. Food. Chem., 48:3990, 2000.
Yi et al., Nature, 452:225-229, 2008.
Young and Anderson, Breast Cancer Res., 10:202, 2008.
Zeng et al., Cancer Res., 62(13):3630-3635, 2002.
Zhang et al., J. Biol. Chem., 275:24436-24443, 2000.
Zhang et al., Proc. Natl. Acad. Sci. USA, 105:7004-7009, 2008.
Zhao et al., J. Biol. Chem., 283(45):31079-31086, 2008.
Zhong et al., Clin. Chen. ACTA., 313:147, 2001.
Zhu et al., Nature., 477:179-184, 2011.
Zweigenbaum et al., J. Pharm. Biomed. Anal., 23(4):723-733, 2000
Zweigenbaum et al., Anal. Chem., 71(13):2294-300, 1999.

1. A method of classifying a subject with triple-negative breast cancer (TNBC) or and BRCA1-related breast cancer as having an active or inactive 53BP1 degradation pathway comprising:
(a) obtaining a sample from said subject comprising tumor cells; and
(b) assessing nuclear protein levels of 53BP1, cathepsin L (CSTL) and vitamin D receptor in said tumor cells,
wherein if (i) the patient has high CTSL, low 53BP1 and low VDR, the 53BP1 degradation pathway is active, (ii) the patient has high CTSL, high 53BP1, and high VDR, the 53BP1 degradation pathway is inactive, and (iii) the patient has low CTSL and low 53BP1, the 53BP1 degradation pathway is inactive and the 53BP1 gene contains a mutation or the transcript is destabilized.

2. The method of claim 1, wherein obtaining comprises taking a sample from said subject.

3. The method of claim 1, wherein said sample is a tumor biopsy or a nipple aspirate.

4. The method of claim 1, further comprising treating said patient with vitamin D and/or a cathepsin inhibitor if the 53BP1 degradation pathway is active.

5. The method of claim 4, further comprising treating said patient with a PARP inhibitor.

6. The method of claim 4, further comprising treating with a cis-platinum agent and/or radiation.

7. The method of claim 1, further comprising treating said patient with a PARP inhibitor, a cisplatinum agent and/or radiation if the 53BP1 degradation pathway is inactive.

8. The method of claim 1, wherein the patient has previously received a PARP inhibitor, a cisplatinum agent and/or radiation.

9. The method of claim 1, wherein the cancer is resistant to a PARP inhibitor, a cisplatinum agent and/or radiation.

10. The method of claim 1, wherein the cancer is recurrent.

11. The method of claim 1, wherein said cancer is metastatic.

12. The method of claim 1, wherein said cancer is TNBC.

13. The method of claim 1, wherein said cancer is BRCA1-related breast cancer.

14. The method of claim 1, further comprising treating the subject if the 53BP1 degradation pathway is inactive and VDR is low.

15. The method of claim 1, wherein assessing comprises ELISA.

16. The method of claim 1, wherein assessing comprises RIA.

17. The method of claim 1, wherein assessing comprises immunohistochemistry.

18. The method of claim 1, further comprising assessing mRNA levels for one or more of CTSL, 53BP1 and VDR.

19. The method of claim 18, wherein assessing comprises RT-PCR.

20. The method of claim 1, further comprising assessing BRCA1 status.


SEQUENCE LISTING (6 sequences)

SEQ ID NO: 1 - DNA, 21 bp, Artificial Sequence
  Synthetic peptide
tcagtacaattaggtgggctt

SEQ ID NO: 2 - DNA, 21 bp, Artificial Sequence
  Synthetic peptide
gagaatcctagagatactgaa

SEQ ID NO: 3 - DNA, 21 bp, Artificial Sequence
  Synthetic peptide
tatagctgttggaaggactag

SEQ ID NO: 4 - DNA, 21 bp, Artificial Sequence
  Synthetic peptide
ccctaagtttacttctctaaa

SEQ ID NO: 5 - DNA, 21 bp, Artificial Sequence
  Synthetic peptide
gcccacctaattgtactgaat

SEQ ID NO: 6 - DNA, 21 bp, Artificial Sequence
  Synthetic peptide
cccacctaattgtactgaatt